(19) United States
(12) Patent Application Publication
(10) Pub. No.: US 2009/0118204 A1
(43) Pub. Date: May 7, 2009

(54) Methods and Compositions Related to Esculentoside A

(75) Inventors: Paul OKUNIEFF, Rochester, NY (US); Lurong ZHANG, Bethesda, MD (US); Zhenyu XIAO, Rochester, NY (US)

Correspondence Address: Ballard Spahr Andrews & Ingersoll, LLP, SUITE 1000, 999 PEACHTREE STREET, ATLANTA, GA, 30309-3915, US

(73) Assignee: UNIVERSITY OF ROCHESTER, Rochester, NY (US)

(21) Appl. No.: 11/791,050

(22) PCT Filed: Nov. 18, 2005
(86) PCT No.: PCT/US2005/042063

§ 371 Date: Sep. 15, 2008

Related U.S. Application Data
(60) Provisional application No. 60/629,449, filed on Nov. 18, 2004.

(51) Int. Cl. A61K 31/704; C07H 15/24

(52) U.S. Cl. 514/33; 536/18.1

(57) ABSTRACT

Disclosed are compositions related to water soluble selective COX-2 inhibitors and methods of using the inhibitors (including Esculentoside A and derivatives thereof).

### CROSS-REFERENCE TO RELATED APPLICATIONS

This application claims benefit of U.S. Provisional Application No. 60/629,449, filed Nov. 18, 2004. The aforementioned application is herein incorporated by this reference in its entirety.

### I. BACKGROUND OF THE INVENTION

A. Field of the Invention

This invention relates generally to methods and compositions related to new selective COX-2 inhibitors.

B. Background Art

Cyclooxygenase is an enzyme that catalyzes the rate-limiting step in the conversion of arachidonic acid to prostaglandins. There are two known types of cyclooxygenase, COX-1 and COX-2. COX-1 is constitutively expressed at low levels in many cell types. Specifically, COX-1 is known to be essential for maintaining the integrity of the gastrointestinal epithelium. COX-2 expression is stimulated by growth factors, cytokines, and endotoxins. The cyclooxygenase 2 isoform is not expressed in most tissues (e.g., liver) under physiological conditions but is highly upregulated under certain conditions. For example, COX-2 is upregulated in inflammatory processes and cancer, for example. Up-regulation of COX-2 is responsible for the increased formation of prostaglandins associated with inflammation. What is needed in the art are novel compositions and methods for inhibiting COX-2.

### II. SUMMARY OF THE INVENTION

In accordance with the purpose(s) of this invention, as embodied and broadly described herein, this invention, in one aspect, relates to a method of reducing radiation damage in a subject comprising administering to the subject an effective amount of a water soluble COX-2 inhibitor.

In another aspect, the invention relates to a method of inhibiting COX-2 in a subject comprising administering to the subject a water soluble COX-2 inhibitor. In yet another aspect, the invention relates to a method of inhibiting a cytokine in a subject comprising administering to the subject a water soluble COX-2 inhibitor. Also disclosed herein is a method of inhibiting PGE2 in a subject comprising administering to the subject a water soluble COX-2 inhibitor. Also disclosed herein is a method of inhibiting nitric oxide (NO) in a subject comprising administering to the subject a water soluble COX-2 inhibitor.

In a further aspect, the invention relates to a method of inhibiting angiogenesis in a subject comprising administering to the subject a water soluble COX-2 inhibitor. Also disclosed herein is a method of inhibiting brain edema in a subject comprising administering to the subject an effective amount of a water soluble COX-2 inhibitor.

Further disclosed herein is a composition comprising a COX-2 inhibiting derivative of EsA.

### III. BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 shows the structure of Esculentoside A (EsA, 3-O—[β-D-glucopyranosyl-(Hensley et al. J Clin Oncol. 17(10):3333-55 (1999 October). Felemovicius et al. Ann Surg. 222(4):504-8 (1995 October), discussion 508-10.)-β-D-xylo-pyranosyl] phytolaccagenin).

FIG. 2 shows alterations of IL-1β in irradiated skin of C3H/HeN mice and its relation with skin damage. The assays were performed with total RNA extracted at different time points from skin irradiated with 30 Gy. FIG. 2A shows an RNase protection assay; FIG. 2B shows quantification by phosphorimaging (folds of increase as compared with control).

FIG. 3 shows alterations of IL-1β in irradiated keratinocytes, vascular endothelium and fibroblasts. The assays were performed with total RNA extracted from different types of cells irradiated with 2.5 or 10 Gy. FIG. 3A shows RNase protection assay; FIG. 3B shows quantification by phosphorimaging system.

FIG. 4 shows reduced skin IR toxicity in IL-1R1 knock-out mice. The C57BL/6 wide type and IL-1R1 knock-out mice were irradiated with 40Gy and the skin score was measured at different time points. Without IL-1R1 signaling, the IR skin damage was reduced at both early stage (FIGS. 4A and B) and late stage (FIG. 4C), indicating that IL1 signaling is critical for IR skin toxicity.

FIG. 5 shows the effect of EsA on skin IR toxicity after 19 days. The mice (5/group) were i.p. injected with 10 mg/kg EsA (as test) or PBS vehicle (as control) or intragastrical administration of 50 mg/kg Celebrex (as positive drug control) 16 hours before and then daily after 30 Gy single dose IR for 4 weeks. The results showed that on day 19, there was a significant difference in the degree of skin damage. While the control mice had a moist desquamation (score above 4.5), the EsA treated mice had only erythema (score about 2) and Celebrex had a score of about 3.

FIG. 6 shows the effect of EsA on skin IR toxicity after 28 days. At the end of the experiment detailed in FIG. 5, (4 weeks after IR), Celebrex lost its protection effect while EsA effectively protected the soft tissue.

FIG. 7 shows the alteration in skin score described in FIG. 6 is statistically significant.

FIG. 8 shows the effect of EsA on reducing or preventing brain edema. C57BL/6 mice were treated with i.p 10 mg/kg EsA or PBS or i.v. 3 mg/kg Dexamethasome 16 hour before the whole heads of mice were irradiated at 40 Gy. After 24 hours, the mice were killed and the brains were harvested, weighed and dried in a 60° C. oven. At the indicated time points (10 min intervals for first two hours), the brains were weighed and the data were expressed as: % water={(wet weight−drying weight)/wet weight}×100. The mice treated with EsA and Dex had less extensive brain edema (P<0.05).

FIG. 9 shows the effect of EsA on reducing or preventing brain edema is statistically significant.

FIG. 10 shows the inhibitory effect of EsA on VEGF production by mouse fibroblast L-929.

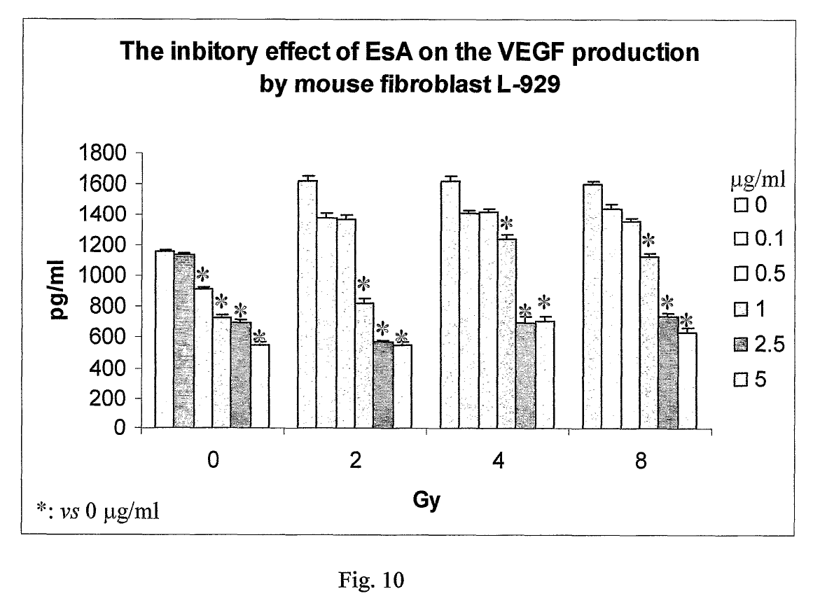

FIG. 11 shows the effect of EsA as on tumor growth. Lewis' lung carcinoma cells were inoculated in syngeneic C57BL/6 mice and treated with or without EsA or Celebrex at the same dose used in sort tissue protection daily for 20 days. The results indicated that EsA had little effect on tumor growth, i.e., neither stimulation nor inhibition of tumor growth, showing that it is safe to use in cancer patients for protecting normal tissue while not promoting tumor growth.

FIG. 12 shows a sensitive reporter system for the hormone transactivity of glucocorticoids receptor (GR) and androgen receptor (AR). The experiment was set up as elucidated in the chart. E8.2.A3 cells derived from L cells (mouse fibroblasts, 41) lack GR, but contain high levels of AR. The cells were cotransfected with wild type mouse GR expression vector (pmGR), reporter vector pMTVCAT and a selection vector pSV2neo vector at a ratio of 30 μg:5 μg:0.5 μg in 100 mm dish using the calcium phosphate precipitation method. Individual clones with stably transfected mGR and CAT reporter gene were selected with 400 μg/ml of G418 in DMEM medium containing 3% charcoal stripped new born calf serum. Then, the cells were seeded (5×10⁵/well) in 24 well plates and treated without (as negative control) or with 5×10⁻⁷ M of Dexamethasome (Dex) and dihydrotestosteron (DHT) as positive control or with different concentrations of ESA (as test) in triplicate for 44 hours. The cells were observed for morphological changes twice a day. There is no change below 4 mg/ml, and only at the level of 40 mg/ml was the death of cells observed.

FIG. 13 shows the EsA at all the test concentrations from the assay shown in FIG. 12. (from 0, 0.4, 4, 40, 400 ng to 4, 40, 400, 4000, 40000 μg/ml). EsA had neither androgen nor glucocorticoidal hormone transactivity, indicating that the EsA is a non-steroidal substance.

FIG. 14 shows that EsA reduces nitric oxide (NO) production. To determine whether EsA protective effects are mediated by down regulation of free radical production, NO production in Raw264.7, a mouse macrophage cell line that had been irradiated, was examined. The linearized standard curve indicated that the assay was functional (FIG. 14A). The study was carried out in Raw264.7 cells that were irradiated with 0, 2, 4 and 8 Gy. The dose of 4 Gy was found to have the best production of NO (FIG. 14B). At this optimal condition, EsA (0.5 or 5 μg/ml) was added 8 hours before the IR at 4 Gy and 24 hours later, the media was harvested and 100 μl of media was measured for NO content in the form of nitrite. The results showed that the EsA inhibited the IR-induced NO production (FIG. 14C).

FIG. 15 shows that EsA possesses anti-COX-2 activity. The prostanoid product was quantified via enzyme immunoassay (EIA) using a broadly specific antibody that binds to all the major prostaglandin compounds (FIG. 15A). To distinguish the inhibition of COX-1 from COX-2, both ovine COX-1 and human recombinant COX-2 enzymes were used as targets. The EsA was applied to this specific testing system and the results (FIG. 15B) demonstrated that EsA had no effect on COX-1, but inhibited the COX-2 in a dose-dependent manner.

FIG. 16 shows the effect of EsA on production of IL1β. After treated without or with 0.1 or 1 μg/ml EsA for 18 hr, the Raw 264.7 macrophage cells or A431 epidermoid cells were irradiated at different IR doses. The protein level of IL1β was measured by ELISA. At dose of 2-4Gy, IL1β was greatly induced (A), which was reduced by EsA at 0.1 μg/ml (B and C).

FIG. 17 shows the similar alopecia effects of celebrex and EsA. The right leg of the mouse was irradiated at a dose of 30 Gy. The cranial alopecia recovered more quickly and completely after EsA than after treatment with celebrex. The right leg of the mice were also irradiated at a dose of 30 Gy. This caused a radiation dermatitis. Administration of PBS vehicle alone or i.g. celebrex at 50 mg/kg was given for 20 days, and photographed 45 days later. There was substantially less dermatitis in the radiation group. Hair regrowth was seen in the EsA group after two weeks, while alopecia remained for greater than five weeks in the Celebrex group.

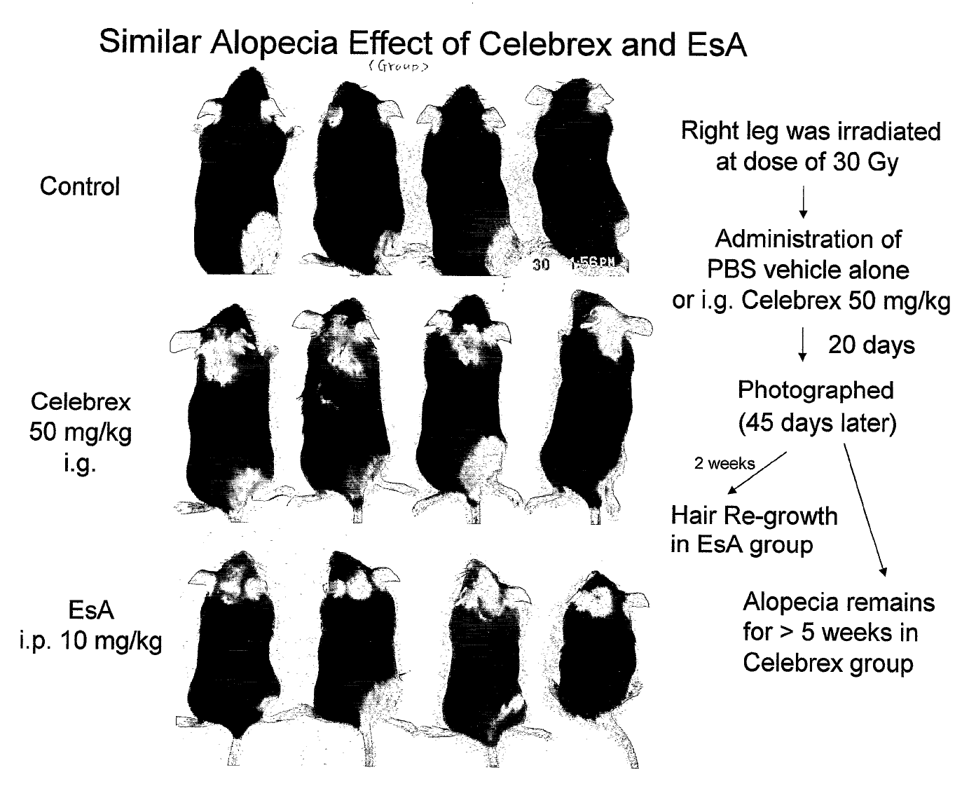

FIG. 18 shows IL-1α production in the skin induced by radiation as effected by EsA.

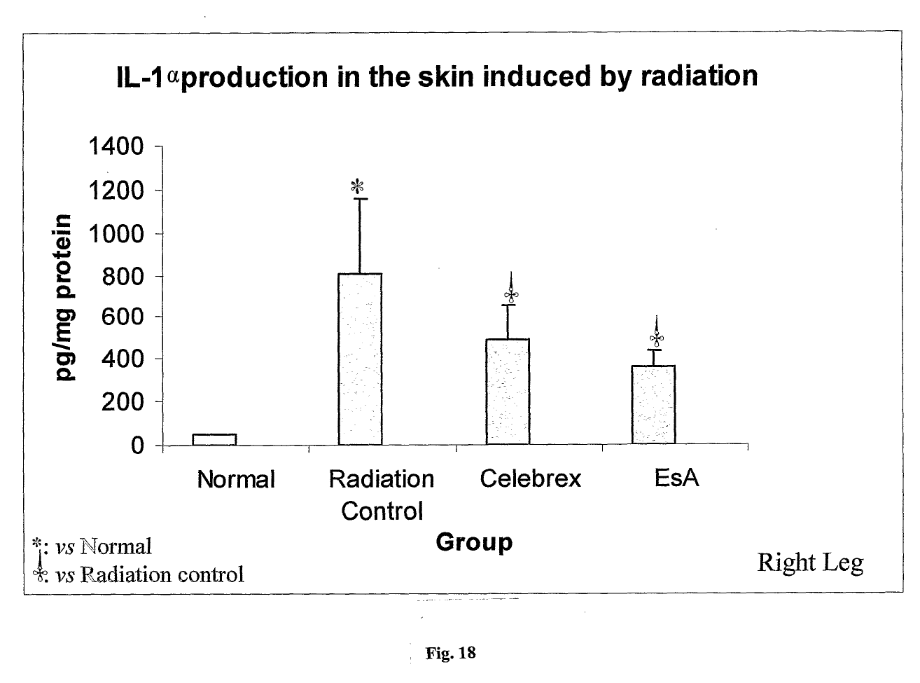

FIG. 19 shows MCP-1 production in the skin induced by radiation as effected by EsA.

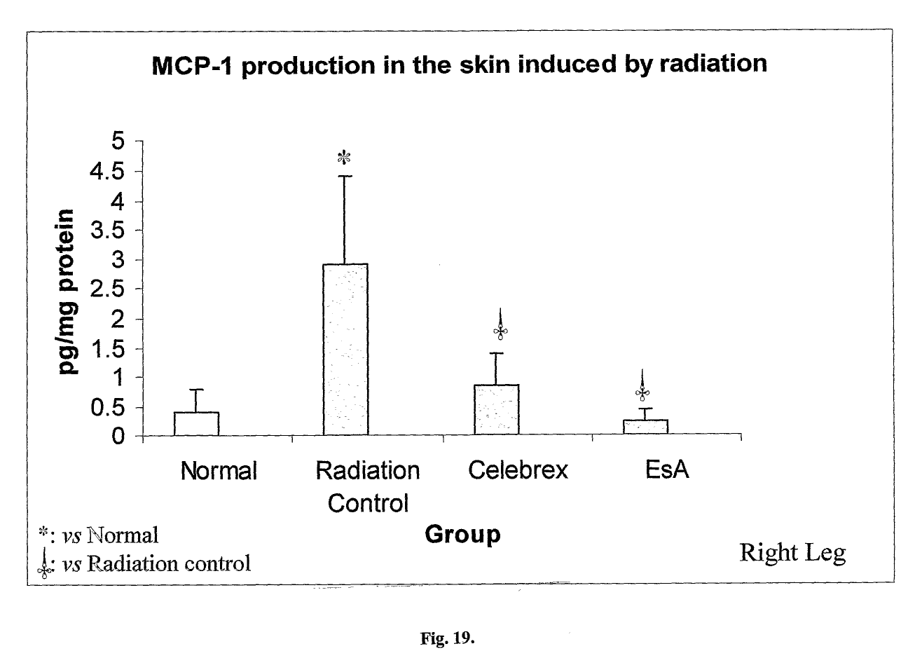

FIG. 20 shows TNF-α production in the skin induced by radiation as effected by EsA.

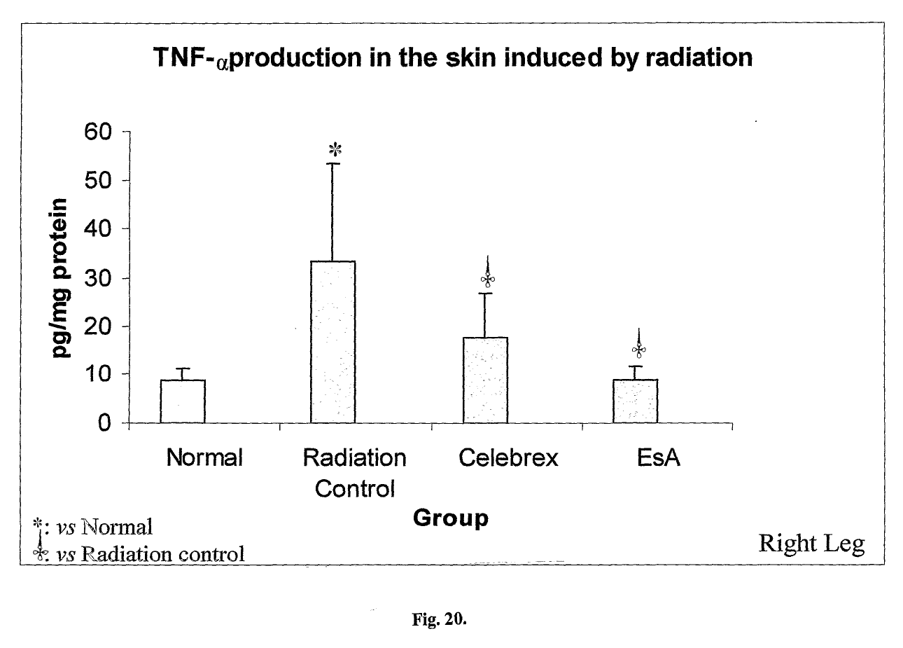

FIG. 21 shows IL-6 production in the skin induced by radiation as effected by EsA.

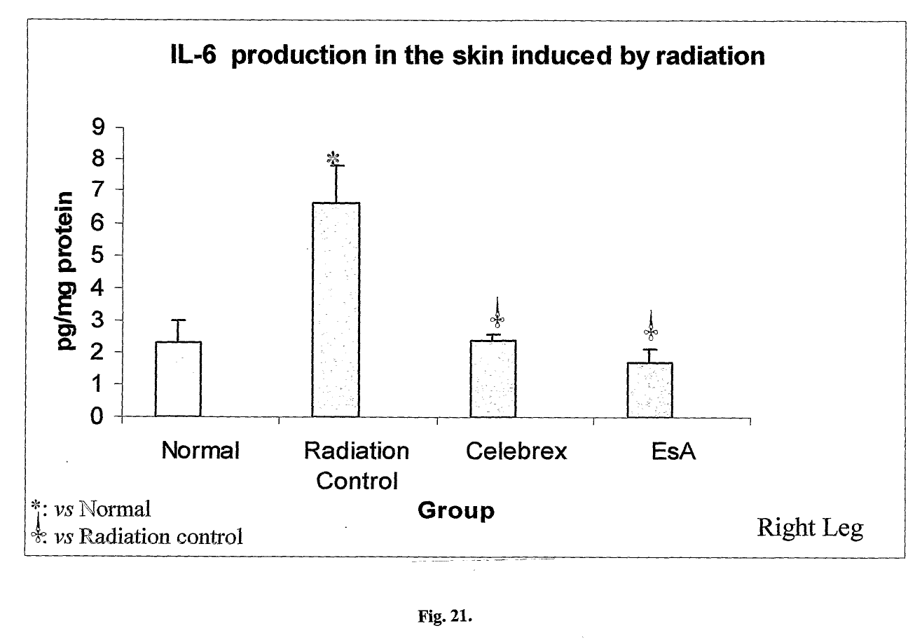

FIG. 22 shows VEGF production in the skin induced by radiation as effected by EsA.

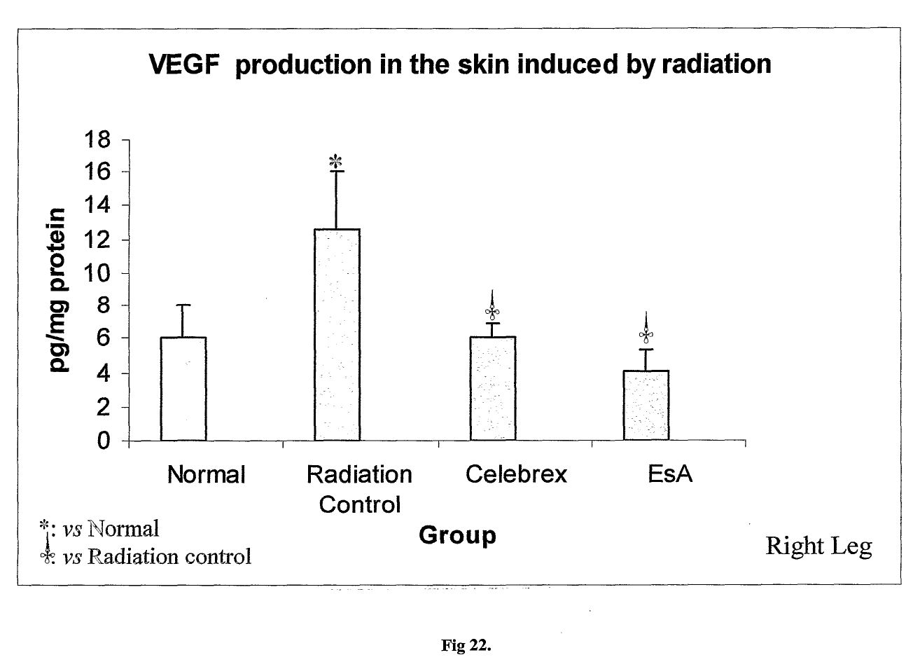

FIG. 23 shows IL1β production in the skin induced by radiation as effected by EsA.

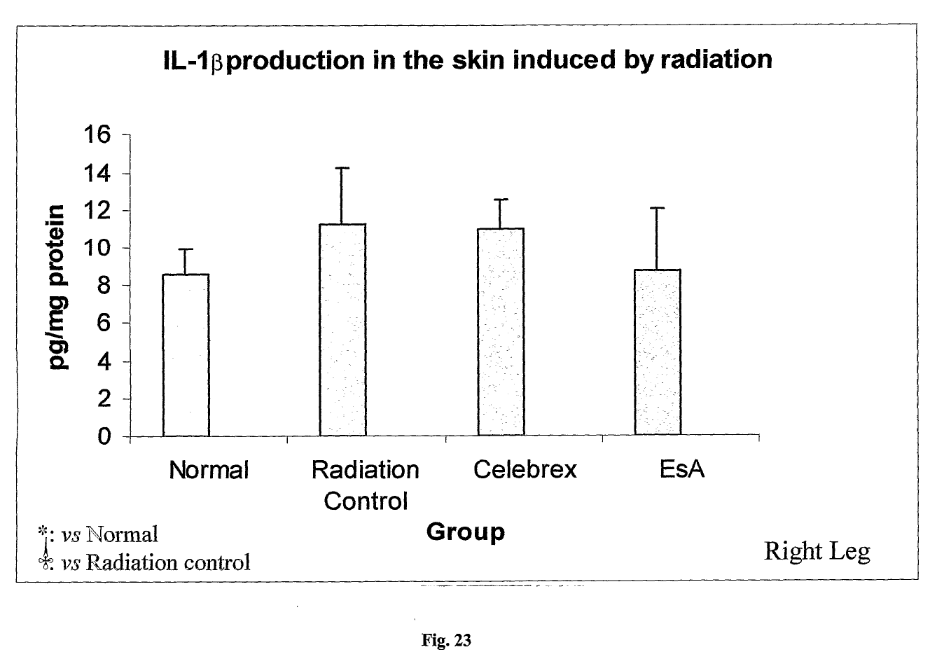

FIG. 24 shows in vivo results of soft tissue fibrosis three months after radiation in control, Celebrex, and EsA groups.

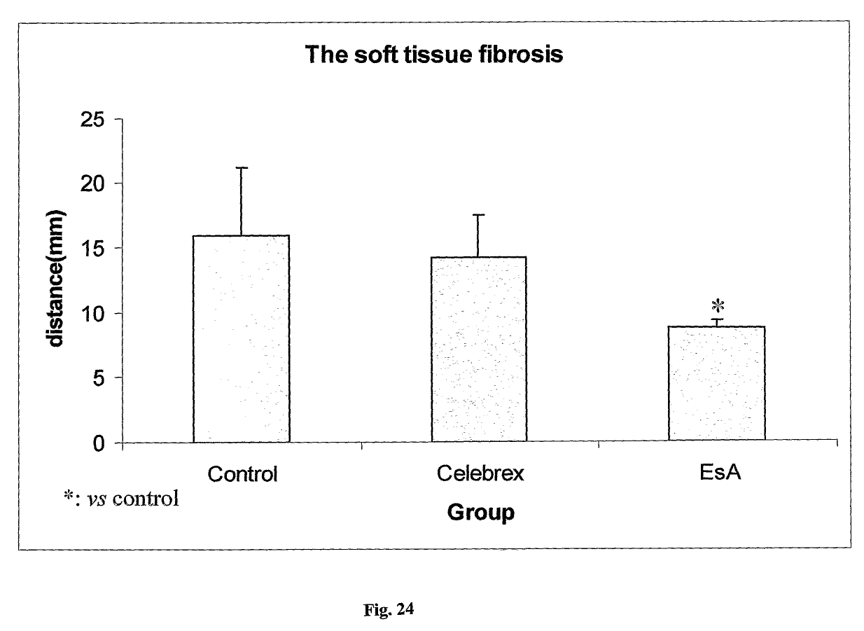

FIG. 25 shows pictures of mice and the results of soft tissue fibrosis three months after radiation in control, Celebrex, and EsA groups.

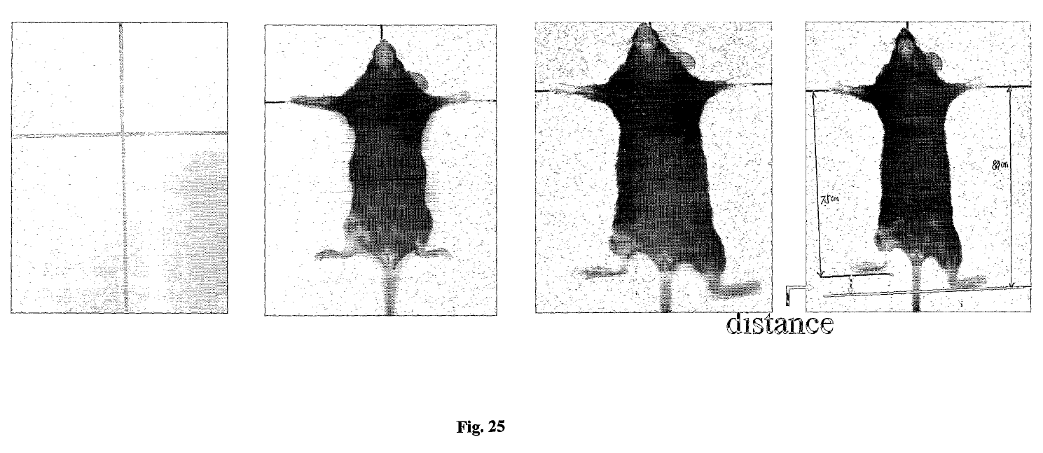

FIG. 26 shows IL1β production by A431 in vitro in epithelial cells.

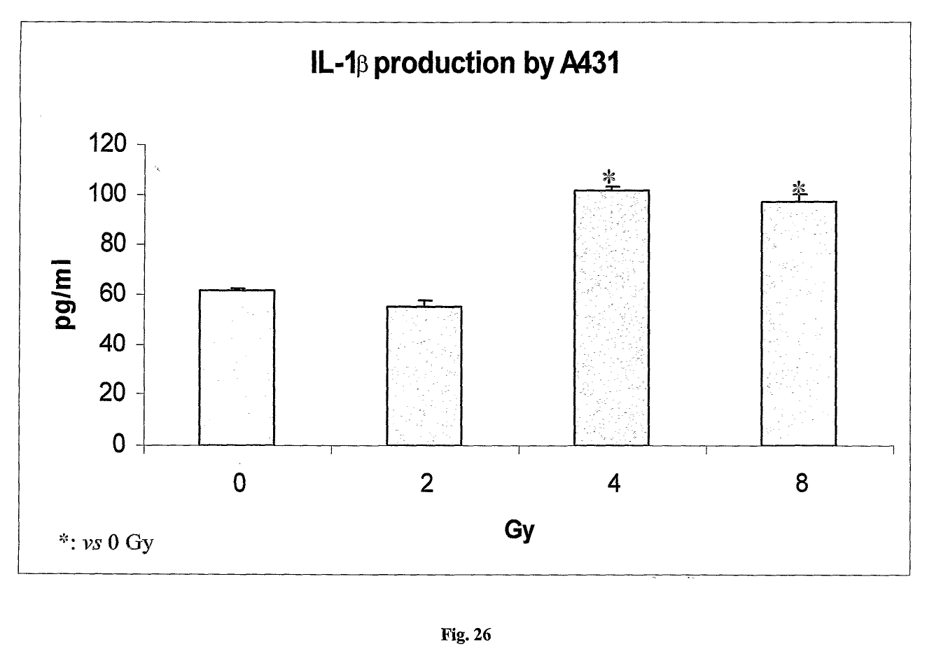

FIG. 27 shows the inhibitory effect on of EsA IL1β production induced by IR in vitro in epithelial cells.

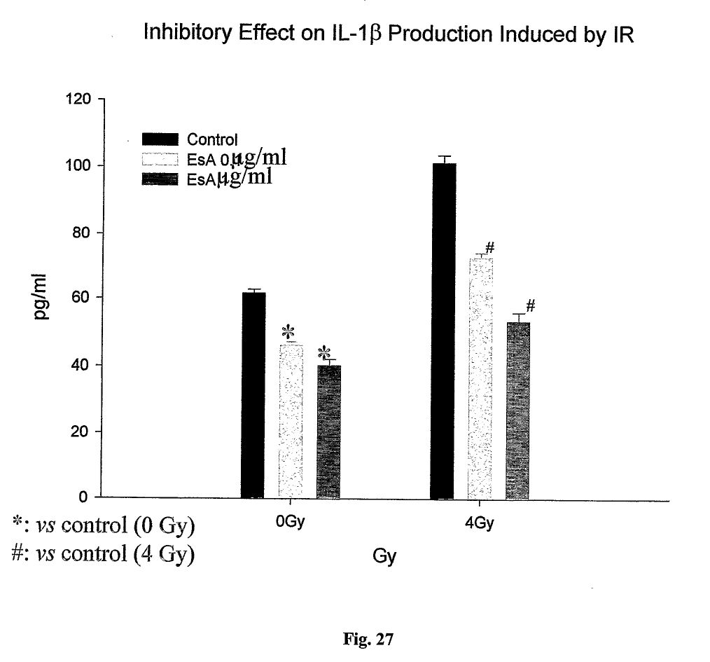

FIG. 28 shows IL-1 production in Raw264.7 cells after radiation and LPS stimulation. Results show in vitro macrophages.

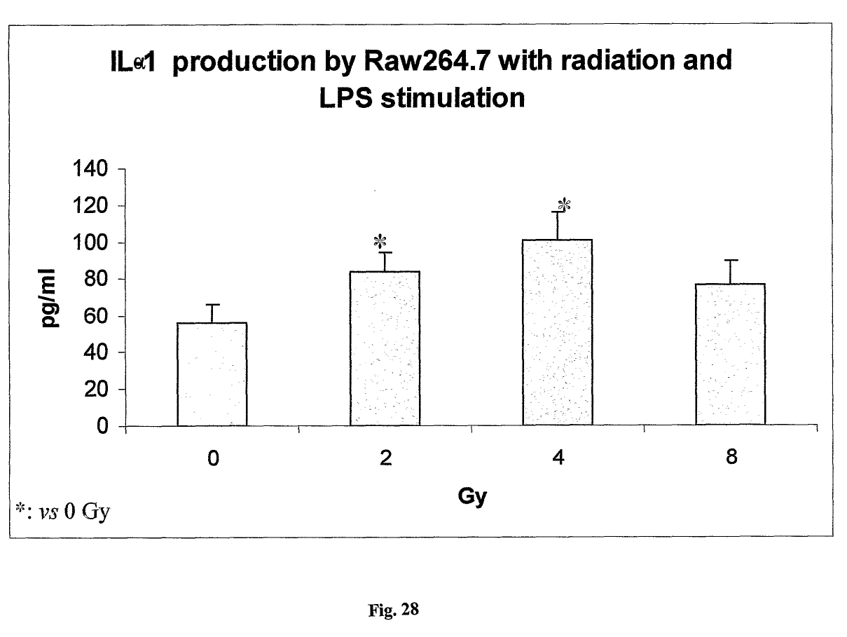

FIG. 29 shows the inhibitory effect of EsA on IL1α production by RAW264.7 in in vitro macrophages.

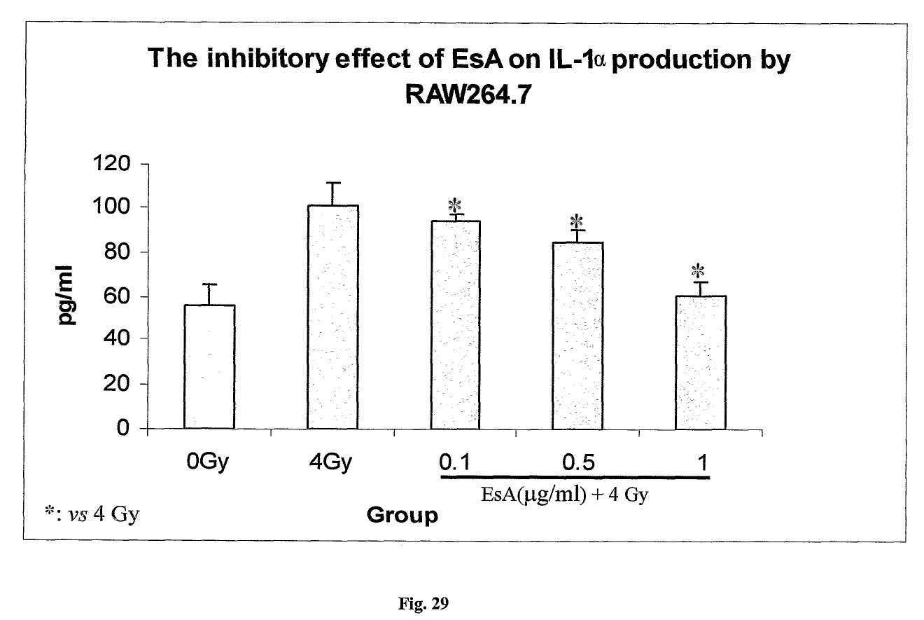

FIG. 30 shows IL-6 production by Raw264.7 with LPS stimulation in in vitro macrophages.

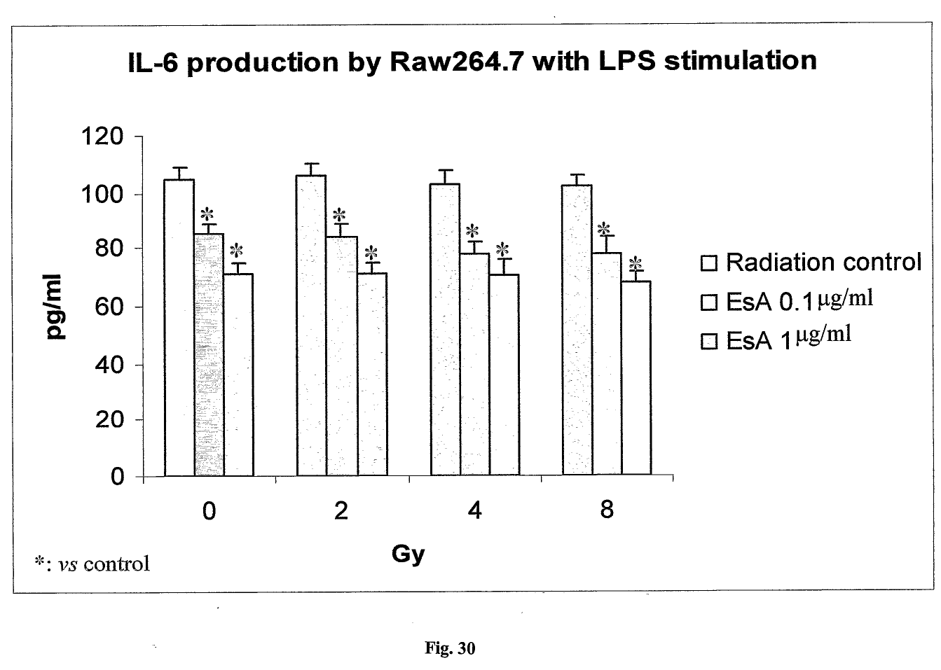

FIG. 31 shows the inhibitory effect of EsA on TNF production by Raw264.7 with LPS and radiation in in vitro macrophages.

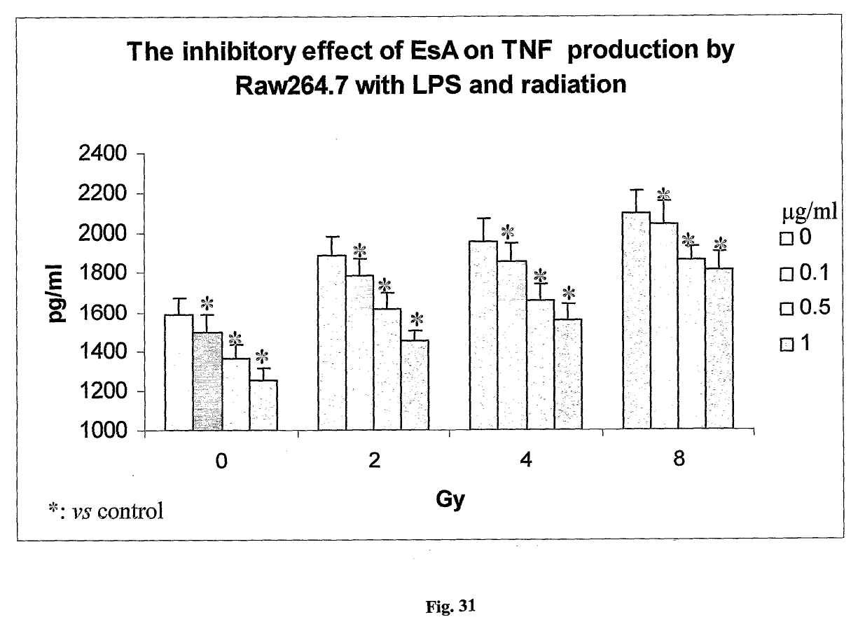

FIG. 32 shows the inhibitory effect on TNF production by mouse fibroblast L-929 in in vitro fibroblasts.

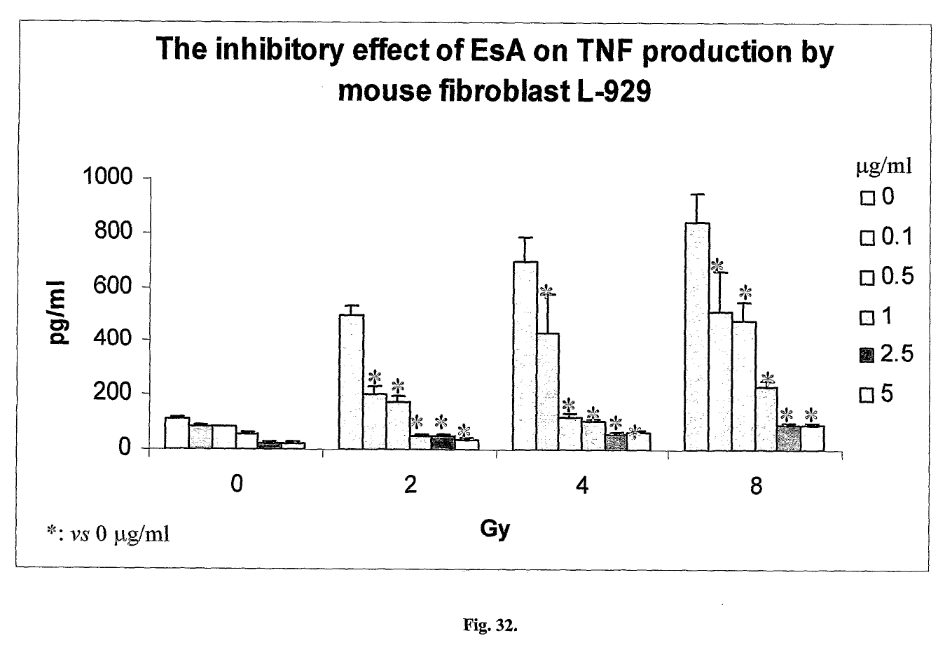

FIG. 33 shows the inhibitory effect on MCP-1 production by mouse fibroblast L-929 in in vitro fibroblasts.

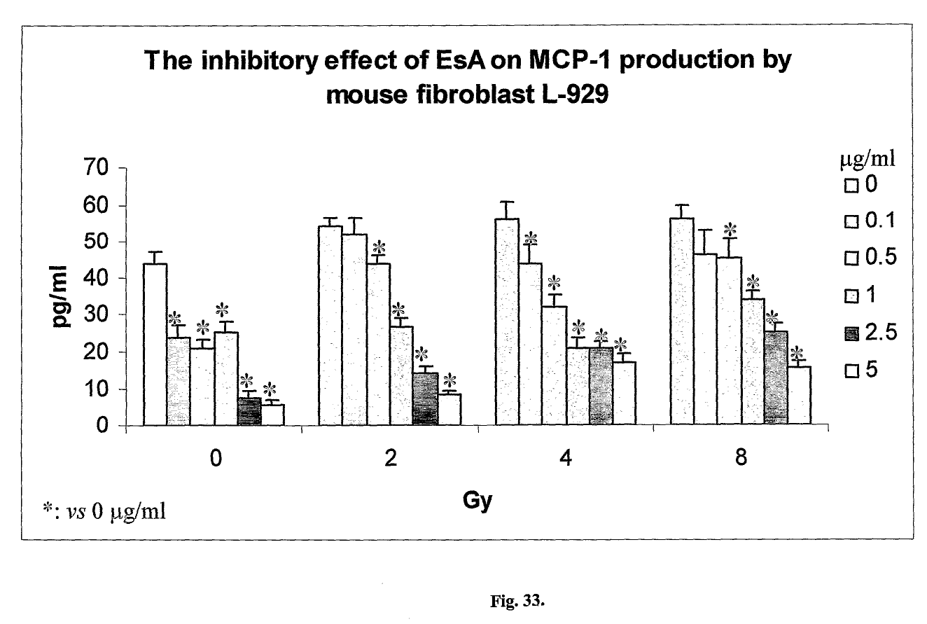

### IV. DETAILED DESCRIPTION OF THE INVENTION

The present invention may be understood more readily by reference to the following detailed description of preferred embodiments of the invention and the Examples included therein and to the Figures and their previous and following description.

As used in the specification and the appended claims, the singular forms “a,” “an” and “the” include plural referents unless the context clearly dictates otherwise. Thus, for example, reference to “a small molecule” includes mixtures of one or more small molecules, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The terms “higher,” “increases,” “elevates,” or “elevation” refers to levels above control levels. The terms “low,” “lower,” “reduces,” or “reduction” refers to levels below control levels. For example, control levels can be normal in vivo levels prior to, or in the absence of, inflammation or the addition of an agent which causes inflammation.

“Inflammation” or “inflammatory” is defined as the reaction of living tissues to injury, infection, or irritation. Anything that stimulates an inflammatory response is said to be inflammatory.

“Inflammatory disease” is defined as any disease state associated with inflammation. The inflammation can be associated with an inflammatory disease. Examples of inflammatory disease include, but are not limited to, asthma, systemic lupus erythematosus, rheumatoid arthritis, reactive arthritis, spondylarthritis, systemic vasculitis, insulin dependent diabetes mellitus, multiple sclerosis, experimental allergic encephalomyelitis, Sjögren's syndrome, graft versus host disease, inflammatory bowel disease (including Crohn's disease and ulcerative colitis) and scleroderma, myasthenia gravis, Guillain-Barré disease, primary biliary cirrhosis, hepatitis, hemolytic anemia, uveitis, Grave's disease, pernicious anemia, thrombocytopenia, Hashimoto's thyroiditis, oophoritis, orchitis, adrenal gland diseases, anti-phospholipid syndrome, Wegener's granulomatosis, Behcet's disease, polymyositis, dermatomyositis, multiple sclerosis, vitiligo, ankylosing spondylitis, Pemphigus vulgaris, psoriasis, and dermatitis herpetiformis.

“Infectious process” is defined as the process by which one organism is invaded by any type of foreign material or another organism. The results of an infection can include growth of the foreign organism, the production of toxins, and damage to the host organism.

“Cancer therapy” is defined as any treatment or therapy useful in preventing, treating, or ameliorating the symptoms associated with cancer. Cancer therapy can include, but is not limited to, apoptosis induction, radiation therapy, and chemotherapy.

“Transplant” is defined as the transplantation of an organ or body part from one organism to another.

“Transplant rejection” is defined as an immune response triggered by the presence of foreign blood or tissue in the body of a subject. In one example of transplant rejection, antibodies are formed against foreign antigens on the transplanted material.

Herein, “inhibition” or “suppression” means to reduce at least one activity as compared to a control (e.g., activity in the absence of such inhibition). It is understood that inhibition or suppression can mean a slight reduction in activity to the complete ablation of all activity. Inhibition or suppression also includes prevention. An “inhibitor” or “suppressor” can be anything that reduces the targeted activity, or has the potential to reduce the targeted activity in the preventative sense. For example, inhibition of COX-2 by a composition such as an EsA or a derivative thereof can be determined by assaying the amount of COX-2 activity present in a cell. The composition can be administered to the cell before it is exposed to circumstances that would cause an elevation in COX-2 activity, and the levels of COX-2 activity can be measured before and after the exposure. In this example, if the amount of COX-2 activity is reduced in the presence of the composition as compared to the amount of COX-2 activity in the absence of the composition, the composition can be said to inhibit COX-2 activity.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

Provided herein are water soluble COX-2 inhibitors (including saponins and derivatives) useful as selective inhibitors of COX-2. These compositions are useful in reducing radiation damage, cytokine inhibition by radiation damage, brain edema, pain, and inflammation, for example. Because these compositions are water soluble, they offer advantages over currently available COX-2 inhibitors. Furthermore, these compositions offer additional advantages over known COX-2 inhibitors. Saponins are a large family of naturally occurring glycoconjugate compounds with considerable structural diversity. Saponins are glycosidic natural plant products, composed of a ring structure (the aglycone) to which is attached one or more sugar chains. The saponins are grouped together based on several common properties. In particular, saponins are surfactants which display hemolytic activity and form complexes with cholesterol. Although saponins share these properties, they are structurally diverse. In particular, the aglycone can be a steroid, triterpenoid or a steroidal alkaloid and the number of sugars attached to the glycosidic bonds vary greatly.

Saponins have been used in pharmaceutical compositions for a variety of purposes. For example, U.S. Pat. No. 5,118,671, describes the use of aescin, a saponin obtained from Aesculus hippocastanum seeds, in pharmaceutical and cosmetic compositions as an anti-inflammatory. Similarly, U.S. Pat. No. 5,147,859, discusses the use of Glyccyrrhiza glabra saponin/phospholipid complexes as anti-inflammatory and anti-ulcer agents and U.S. Pat. No. 5,166,139, describes the use of complexes of saponins and aglycons, obtained from Centella asiatica and Terminalia sp., with phospholipids in pharmaceutical compositions. International Publication No. WO 91/04052, published 4 Apr. 1991, discusses the use of solid Quillaja saponaria saponin/GnRH vaccine compositions for immunocastration and immunospaying.

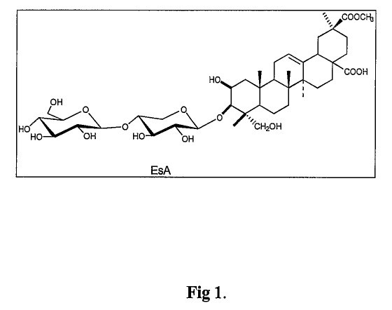

The saponin family includes Esculentosides A, B, C, D and E, and are isolated from Phytolacca esculent. Esculentoside A (EsA, 3-O—[β-D-glucopyranosyl-(Hensley et al. (1999); Felemovicius et al. (1995))-β-D-xylo-pyranosyl] phytolaccagenin, FIG. 1) is a highly purified saponin from Phytolacca esculent. EsA has a molecular weight of 826 Daltons (Yi et al. Chinese Herb Medicine, 15 (2): 7-11 (1984)). Because of its hydrophilic radices such as hydroxide and carboxyl, EsA has a high water-solubility. Derivatives and analogs of EsA are also contemplated and are discussed below. Esculentoside A does not have a cross reaction with sulfonamide antibiotics, unlike many non-steroidal anti-inflammatory drugs (NSAIDS). EsA has anti-inflammatory effects with mechanisms differing from currently used anti-inflammatory drugs.

Inflammation is a complex stereotypical reaction of the body expressing the response to damage of its cells and vascularized tissues. The discovery of the detailed processes of inflammation has revealed a close relationship between inflammation and the immune response. The main features of the inflammatory response are vasodilation, i.e. widening of the blood vessels to increase the blood flow to the infected area; increased vascular permeability, which allows diffusible components to enter the site; cellular infiltration by chemotaxis, or the directed movement of inflammatory cells through the walls of blood vessels into the site of injury; changes in biosynthetic, metabolic, and catabolic profiles of many organs; and activation of cells of the immune system as well as of complex enzymatic systems of blood plasma.

There are two forms of inflammation, acute and chronic. Acute inflammation can be divided into several phases. The earliest, gross event of an inflammatory response is temporary vasoconstriction, i.e. narrowing of blood vessels caused by contraction of smooth muscle in the vessel walls, which can be seen as blanching (whitening) of the skin. This is followed by several phases that occur over minutes, hours and days later. The first is the acute vascular response, which follows within seconds of the tissue injury and lasts for several minutes. This results from vasodilation and increased capillary permeability due to alterations in the vascular endothelium, which leads to increased blood flow (hyperemia) that causes redness (erythema) and the entry of fluid into the tissues (edema).

Examples of chronic inflammatory diseases include tuberculosis, chronic cholecystitis, bronchiectasis, rheumatoid arthritis, Hashimoto's thyroiditis, inflammatory bowel disease (ulcerative colitis and Crohn's disease), silicosis and other pneumoconiosis, and implanted foreign body in a wound.

Activated cells can also be identified at the site of inflammation. “Activated cells” are defined as cells that participate in the inflammatory response. Examples of such cells include, but are not limited to, T-cells and B-cells, macrophages, NK cells, mast cells, eosinophils, neutrophils, Kupffer cells, antigen presenting cells, as well as vascular endothelial cells.

Macrophages release cytokines (e.g., tumor necrosis factor, interleukin-1), which heighten the intensity of inflammation by stimulating inflammatory endothelial responses; these endothelial changes help recruit large numbers of T cells to the inflammatory site.

Damaged tissues release pro-inflammatory mediators (e.g., Hageman factor (factor XII) that trigger several biochemical cascades. The clotting cascade induces fibrin and several related fibrinopeptides, which promote local vascular permeability and attract neutrophils and macrophages. The kinin cascade principally produces brakykinin, which promotes vasodilation, smooth muscle contraction, and increased vascular permeability.

Disclosed herein are methods of treating inflammation in a subject by administering to the subject an effective amount of a water soluble COX-2 inhibitor. Such an inhibitor can be a saponin, such as EsA or a derivative thereof. Optionally, the inhibitor is not EsA. In various kinds of animal inflammatory models, EsA shows a strong inhibition of inflammation. In acute inflammation models, EsA markedly lowered the vascular permeability induced by 0.7% acetic acid in mice and the swelling of murine ears induced by zylene. EsA also inhibited the swelling of rat hind paws induced by carrageenan. The effects lasted for more than 5 hours. Furthermore, in a chronic inflammation model, the proliferation of granuloma induced by cotton pellet was significantly inhibited. EsA also suppressed the swelling of adrenalectomised rat hind paws induced by carrageenan, which shows that the anti-inflammatory property of EsA was not dependent on the pituitary-adrenal system. In an autoimmunity animal model, EsA decreased the inflammation of joint and viscera, and ameliorated the symptoms such as the destruction of cartilage.

The molecular mechanism is associated with the reduction of several key inflammatory mediators. In vivo, EsA dose-dependently decreased the TNF, IL-1 and IL-6 levels in the sera of mice following LPS challenge. In vitro, EsA or a derivative thereof significantly reduced the release of TNF, IL-1 and IL-6 from the peritoneal macrophages derived from mice pretreated with thioglycolate. EsA also suppressed LPS-induced high expression of adhesion molecular such as ICAM-1 and CD18, which play a vital role in the extravasations of neutrophils in the inflammatory process. Furthermore, EsA diminished the functions of activated macrophages such as phagocytosis and antibody production and secretion of cytokines (Ju et al. Pharmacology 56(4):187-95 (1998 April); Ju et al. Yao Xue Xue Bao. 29(4):252-5 (1994). In another example, EsA markedly decreased serum hemolysin concentration in sensitized mice challenged with sheep red blood cells. EsA also accelerated the apoptosis of activated thymocytes and inhibited the production of IL-2 from activated splenocytes, showing that EsA can act as an immunological modulator.

Disclosed are methods of inhibiting a cytokine in a subject comprising administering to the subject a water soluble COX-2 inhibitor. The cytokine can be selected from the group consisting of angiogenic, growth, fibrogenic, and inflammatory cytokines. Examples of such cytokines include, but are not limited to, IL1, IL6, TNFα, TGFβ, VEGF, and MCP1 or any combination thereof. The COX-2 inhibitor can be administered in a variety of ways, as disclosed herein. Examples include intraarticularly, intravenously, intrathecally, intramuscularly, subcutaneously, transdermally, and orally. They may also be administered by rectal suppository, inhaler, or intraoperative wash. Other examples of methods of administration are disclosed below. Examples of water soluble COX-2 inhibitors include saponins, such as EsA, or a COX-2 inhibiting derivative thereof. Examples of derivatives of EsA can also be found below.

“Inhibiting a cytokine” refers to blocking or reducing at least one cytokine mediated event.

Also disclosed are methods of inhibiting PGE₂ in a subject comprising administering to the subject a soluble COX-2 inhibitor. Also disclosed are methods of inhibiting nitric oxide (NO) in a subject comprising administering to the subject a water soluble COX-2 inhibitor. EsA inhibited the production of prostaglandin E₂ (PGE₂), platelet-activating factor (PAF), and nitric oxide (NO). PGE₂ is known to play a major role in acute inflammation. Nitric Oxide has a key role in perpetual inflammation (Fang et al. Yao Xue Xue Bao. 26(10):721-4 (1991)).

Inflammation can be associated with a number of different diseases and disorders. Examples of inflammation include, but are not limited to, inflammation associated with hepatitis, inflammation associated with the lungs, and inflammation associated with an infectious process. Inflammation can also be associated with liver toxicity, which can be associated in turn with cancer therapy, such as apoptosis induction or chemotherapy, or a combination of the two, for example.

When the inflammation is associated with an infectious process, the infectious process can be associated with a viral infection. Examples of viral infections include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian immunodeficiency virus, Human immunodeficiency virus type-1, and Human immunodeficiency virus type-2.

The infectious process can also be associated with a bacterial infection. Examples of bacterial infections include, but are not limited to, M. tuberculosis, M. bovis, M bovis strain BCG, BCG substrains, M avium, M intracellulare, M africanum, M kansasii, M. mariun, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

The infectious process can also be associated with a parasitic infection. Examples of parasitic infections include, but are not limited to, Toxoplasma gondii, Plasmodium species such as Plasmodium falciparum, Plasmodium vivax, Plasmodium malariae, and other Plasmodium species, Trypanosoma brucei, Trypanosoma cruzi, Leishmania species such as Leishmania major, Schistosoma such as Schistosoma mansoni and other Shistosoma species, and Entamoeba histolytica.

The infectious process can also be associated with a fungal infection. Examples of fungal infections include, but are not limited to, Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

The inflammation can be associated with cancer. Examples of types of cancer include, but are not limited to, lymphoma (Hodgkins and non-Hodgkins) B-cell lymphoma, T-cell lymphoma, leukemia such as myeloid leukemia and other types of leukemia, mycosis fungoide, carcinoma, adenocarcinoma, sarcoma, glioma, blastoma, neuroblastoma, plasmacytoma, histiocytoma, melanoma, adenoma, hypoxic tumor, myeloma, AIDS-related lymphoma or AIDS-related sarcoma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, head and neck carcinoma, hematopoietic cancer, testicular cancer, colo-rectal cancer, prostatic cancer, and pancreatic cancer.

Also disclosed are methods of reducing transplant rejection in a recipient by administering to the recipient a water soluble COX-2 inhibitor. Inflammation is associated with transplant rejection in a transplant recipient. In one example of transplant rejection, antibodies are formed against foreign antigens on the transplanted material. The transplantation can be, for example, organ transplantation, such as liver, kidney, skin, eyes, heart, or any other transplantable organ of the body or part thereof.

Transplantation immunology refers to an extensive sequence of events that occurs after an allograft or a xenograft is removed from a donor and then transplanted into a recipient. Tissue is damaged at both the graft and the transplantation sites. An inflammatory reaction follows immediately, as does activation of biochemical cascades. A series of specific and nonspecific cellular responses ensues as antigens are recognized. Antigen-independent causes of tissue damage (i.e., ischemia, hypothermia, reperfusion injury) are the result of mechanical trauma as well as disruption of the blood supply as the graft is harvested. In contrast, antigen-dependent causes of tissue damage involve immune-mediated damage.

Rejection is the consequence of the recipient's alloimmune response to the nonself antigens expressed by donor tissues. In hyperacute rejection, transplant subjects are serologically presensitized to alloantigens (i.e., graft antigens are recognized as nonself). Histologically, numerous polymorphonuclear leukocytes (PMNs) exist within the graft vasculature and are associated with widespread microthrombin formation and platelet accumulation. Little or no leukocyte infiltration occurs. Hyperacute rejection manifests within minutes to hours of graft implantation. Hyperacute rejection has become relatively rare since the introduction of routine pretransplantation screening of graft recipients for antidonor antibodies.

In acute rejection, graft antigens are recognized by T cells; the resulting cytokine release eventually leads to tissue distortion, vascular insufficiency, and cell destruction. Histologically, leukocytes are present, dominated by equivalent numbers of macrophages and T cells within the interstitium. These processes can occur within 24 hours of transplantation and occur over a period of days to weeks.

In chronic rejection, pathologic tissue remodeling results from peritransplant and posttransplant trauma. Cytokines and tissue growth factor induce smooth muscle cells to proliferate, to migrate, and to produce new matrix material. Interstitial fibroblasts are also induced to produce collagen. Histologically, progressive neointimal formation occurs within large and medium arteries and, to a lesser extent, within veins of the graft. Leukocyte infiltration usually is mild or even absent. All these result in reduced blood flow, with subsequent regional tissue ischemia, fibrosis, and cell death. (Prescilla et al. http://www.emedicine.com, Immunology of Transplant Rejection, updated Jun. 20, 2003).

Transplant rejection may occur within 1-10 minutes of transplantation, or within 10 minutes to 1 hour of transplantation, or within 1 hour to 10 hours of transplantation, or within 10 hours to 24 hours of transplantation, within 24 hours to 48 hours of transplantation, within 48 hours to 1 month of transplantation, within 1 month to 1 year of transplantation, within 1 year to 5 years of transplantation, or even longer after transplantation.

Disclosed herein are methods of treating radiation damage. Ionizing radiation (IR) remains a main stream therapy for cancer, since it controls both primary and metastatic cancer without significant systemic damage. However, radiation therapy does cause IR-induced local damage of normal tissue (radiation toxicity), leading to a temporary or persistent impairment of irradiated tissues, which lowers the life quality of cancer patients. Some severe side effects can even result in the discontinuation of the life-saving radiation therapy (Johansen et al. Radiother Oncol. 40: 101-9 (1996), Niemierko et al. Int J Radiat Oncol Biol Phys. 25: 135-45, 1993., Wiess et al. Toxicology 15; 189(1-2):1-20 (2003 July) Protection against ionizing radiation by antioxidant nutrients and phytochemicals. Toxicology. 15:189(1-2):1-20 (2003 July), Goiten et al. Cancer 55: 2234-9 (1985)). Radiation damage can also occur by exposure to nuclear radiation, or exposure to a weapon that causes radiation.

Disclosed herein are methods of reducing radiation damage in a subject comprising administering to the subject an effective amount of a water soluble COX-2 inhibitor. As disclosed above, the radiation damage can be caused by radiation therapy, such as that used to treat cancer. The radiation damage can also be caused by nuclear radiation, or by a weapon, such as a terrorist agent. The compositions herein can be administered prior to, after, or during exposure to radiation.

Radiation toxicity can be divided into two main stages: early toxicity and late toxicity (MacKay et al. Radiother Oncol. 46:215-6 (1998), Rubin et al. Radiother Oncol. 35: 9-10, (199?), Dubray et al. Cancer Radiother. 1: 744-52 (1997), Vozenin-Brotons et al. Radiat Res. 152: 332-7 (1999), Lefaix et al. Br J Radiol Suppl. 19: 109-13 (1986), Lefaix et al. Soc Biol Fil. 191: 777-95 (1997), Verola et al. Br J Radiol Suppl. 19: 104-8 (1986)). For example, in the irradiated soft tissue, there is early radiation dermatitis (ERD) that occurs within one month after IR, and late radiation fibrosis (LRF) which develops two months later. In the irradiated lung, there is pnuemonitis (early) and lung fibrosis (late) (Chen et al. Int J Radiat Oncol Biol Phys. 49: 641-8 (2001), Chen et al. Seminars in Radiation Oncology 12: 26-33 (2002), Marks et al. Int J Radiat Biol. 76: 469-75 (2000)). In the irradiated brain, there is brain edema (early) and brain degeneration (late). The pathophysiological mechanisms underlying these phenomena have been studied, but remain unclear.

In general, IR kills cell through the production of free radicals. The IR toxicity is a result of counteraction of host defense system that responds to IR physical insult. Upon IR, the cells are damaged by free radicals, and undergo either repair or apoptosis/death, which initiates the cascade of signal transduction pathways (such as Nuclear factor-κB (NFKβ, etc.). As a result, IR up-regulates the expression of inflammatory mediators (such as cytokines, lymphokines and chemokines) and immunomodulatory molecules (MHC, co-stimulatory molecules, adhesion molecules, death receptors, heat shock proteins) in irradiated tumor, stromal, and vascular endothelial cells (Friedman et al.). Among them, for example, IL1, IL6, MCP-1, COX-2 and TGF [symbol] play critical roles in IR toxicity (Chen et al. (2001) Hallahan et al. Important Adv Oncol.:71-80 (1993)). The accumulated cytokines and chemokines attract the immune cells (such as macrophages, dendritic cells, T cells and B cells) to the irradiated spot to engulf the apoptotic and necrotic cellular debris. After internalizing the debris, some of the mutated normal tissue “self” antigens can be presented by dendritic cells to T cells (McBride et al. Radiat Res. 162(1):1-19 (2004 July)). The interaction of sensitized T cells with the existing IR-induced mutated “wrong proteins” or “wrong genes” (which can pass to daughter cells) in irradiated normal tissues triggers a new wave of mass production of cytokines, which occurs a few months after IR, which may be the driving force for the late toxicity (chronic inflammation). This process is evidenced by the several waves of mass production of secretory molecules (cytokines and inflammatory mediators) at the stages of early and late toxicity.

A network exists between IR-induced molecules, such as interaction among NO, NF—Kβ, cytokines and COX. An interaction loop and feed-back control exists among these molecules. Upon IR, the NO and the signaling of DNA breakage directly activate NF-kβ, which induces IL1β. The IL1 [symbol] binds to its receptors, which again triggers NFkβ and P38 pathways to enhance its production, a positive feed-back to amplify the inflammation signaling. IL1 [symbol] is a key cytokine in the IR inflammation process. As one of the effects, IL1β enhances the expression of COX-2, and together they markedly induce inflammatory angiogenesis (Kuwano et al. FASEB J. 18(2):300-10 (2004)), a critical process in IR inflammation (toxicity). As a control, IL-1β-induced activation of the COX-2 gene is modulated by NF-kβ (Kirtikara et al. (2000), Crofford et al. Arthritis Rheum. 40, 226-236 (1997)). The COX-2 selective inhibitors can block IL1 [symbol] induced angiogenesis but only partially block VEGF-induced angiogenesis. Similarly, the IL1 [symbol] induced angiogenesis is much less in the COX-2 knockout mice than wild-type mice (Kuwano et al. (2004)). Overexpression of COX-2 also is accompanied by up-regulation of nitric oxide synthases (Tsuji et al. Nippon Rinsho. 56: 2247-2252 (1998)), which can intensify local damage.

Cyclooxygenase is the rate-limiting step in the conversion of arachidonic acid to prostaglandins. There are two known genes of cyclooxygenase, COX-1 and COX-2. COX-1 is constitutively expressed at low levels in many cell types. Specifically, COX-1 is known to be essential for maintaining the integrity of the gastrointestinal epithelium. COX-2 expression is stimulated by growth factors, cytokines, and endotoxins. The cyclooxygenase 2 isoform (COX-2) is not expressed in most tissues (e.g., liver) under physiological conditions but is highly upregulated in inflammatory processes and cancer, for example. Up-regulation of COX-2 is responsible for the increased formation of prostaglandins associated with inflammation.

COX-2 is also associated with cancer. For example, COX-2 is overexpressed in adenocarcinoma (Tsuji et al. (1998), Sano et al. Cancer Res. 55: 3785-3789 (1995), Murata et al. Am. J. Gastroenterol. 94: 451-455 (1999)). The enhanced COX-2-induced synthesis of prostaglandins stimulates cancer cell proliferation (Sheng et al. J. Biol. Chem. 276: 18075-18081 (2001), Achiwa et al. Clin. Cancer Res. 5: 1001-1005 (1999), promotes angiogenesis (Ben-Av et al. FEBS Lett. 372: 83-87 (1995), Tsuji et al. J. Exp. Clin. Cancer Res. 20: 117-129 (2001)), inhibits apoptosis (Sheng et al. Cancer Res. 58:362-366 (1998)) and increases metastatic potential (Kakiuchi et al. (2002), Xue et al. World J. Gastroenterol. 9: 250-253 (2003)). The inhibition of COX-2 has dual benefits: protecting the normal tissues and inhibiting the cancer cells. In addition, COX-2 inhibitors can act as chemopreventive agents. IL1β-stimulated COX-2 expression can be found in almost all types of cells, including monocytes/macrophages (Caivano et al. J. Immunol. 164: 3018-3025 (2000), vascular endothelial cells (Kirtikara et al. (2000)), stromal cells (Bamba et al. Int. J. Cancer 83: 470-475 (1999)), epithelial cells and nonepithelial cells, showing that this interaction is critical for all types of tissue damage/inflammation processes. The blocking of these paired molecules has therefore not been restricted to a specific tissue.

Disclosed herein are methods of inhibiting COX-2 in a subject comprising administering to the subject a water soluble COX-2 inhibitor. Such inhibitors may be administered in a variety of ways. Examples include intraarticularly, intravenously, intrathecally, intramuscularly, subcutaneously, transdermally, and orally. They may also be administered by rectal suppository, inhaler, or intraoperative wash. Other examples of methods of administration are disclosed below. Examples of water soluble COX-2 inhibitors include saponins, such as EsA, or a COX-2 inhibiting derivative thereof. Examples of derivatives of EsA can be found below.

Disclosed herein are methods of treating pain in a subject by administering to the subject an effective amount of EsA or a derivative thereof. Pain is often associated with inflammation and the presence of COX-2. EsA and derivatives thereof can be used as an analgesic for pain management.

Also contemplated are methods of inhibiting or preventing brain edema in a subject comprising administering to the subject an effective amount of a water soluble COX-2 inhibitor. Cerebral edema occurs due to an increase in brain water content. It can be either intracellular or extracellular. Intracellular edema is defined by cellular swelling, usually of astrocytes, and classically is seen following cerebral ischemia caused by cardiac arrest or head injury. The blood brain barrier is intact. Extracellular edema is a consequence of vascular injury with disruption of the blood brain barrier. Causes include trauma, tumor, and abscess. Ultimately, these changes can lead to herniation. Brain edema can also be radiation induced.

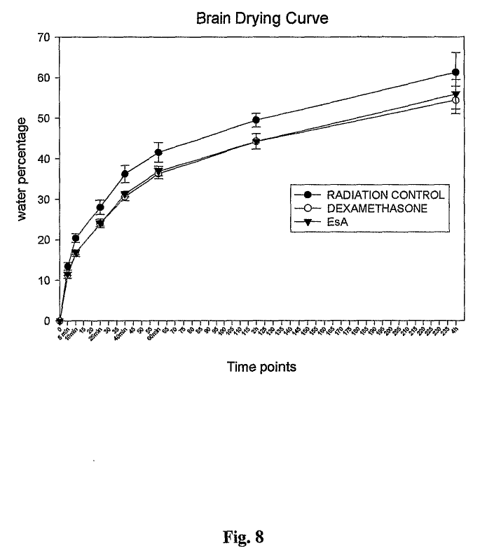

Example 4 shows that EsA has a strong inhibitory effect on erythema and can reduce IR-induced brain edema. The results, as seen in FIG. 8, show that the brains of irradiated mice without EsA had a higher water percentage than those treated with EsA or Dexamethasone.

Disclosed are methods of inhibiting angiogenesis in a subject comprising administering to the subject a water soluble COX-2 inhibitor. An increase in the expression of COX-2 has been correlated with a poor clinical outcome in patients with colorectal and other cancers. It has been shown that the COX-2 expressed in the epithelial cell compartment regulates angiogenesis in the stromal tissues of the mammary gland and that it is critical during mammary cancer progression (Chang et al. PNAS, DOI:10.1073/pnas.2535911100, Dec. 15, 2003.).

The effect of inhibition of prostanoid synthesis on COX-2 transgenic mice was determined, using a strain that develops spontaneous mammary tumors. It was observed that indomethacin strongly decreased microvessel density and inhibited tumor progression. Indomethacin also inhibited upregulation of angiogenic regulatory genes in COX-2 transgenic mammary tissue. In addition, it was shown that prostaglandin E2 stimulated the expression of angiogenic regulatory genes in mammary tumor cells isolated from COX-2 transgenic mice and treated with celecoxib, a COX-2-specific inhibitor, and reduced tumor growth and microvessel density.

The EsA molecule and its derivatives without sidechains (as described below) has molecular similarity to steroid hormones. This molecule can block enzymes related to steroid metabolism and interconversion. An example of such an enzyme is the aromatase enzyme, which converts androgens to estrogens. Agents that block this enzyme are the preferred treatment for many women with post-menopausal breast cancer. As disclosed herein, EsA and its derivatives can have anti-inflammatory effects, which can be related to steroid effects and include cytokine-modifying effects. EsA and its derivatives can also have a therapeutic effect on the endometritis and breast adenoma (two types of estrogen related chronic diseases).

Disclosed herein and useful in the methods described are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that, while specific reference of each various individual and collective combinations and permutation of these compounds may not be explicitly disclosed, each is specifically contemplated and described herein. For example, if a particular molecule, such as EsA, is disclosed and discussed and a number of modifications that can be made to a number of places within the molecule can be made, specifically contemplated is each and every combination and permutation of the molecule unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxamate, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkylalcohol” specifically refers to an alkyl group that is substituted with one or more hydroxyl groups, as described below. The term “alkylthiol” specifically refers to an alkyl group that is substituted with one or more thiol groups, as described below. The term “alkylalkoxy” specifically refers to an alkyl group that is substituted with one or more alkoxy groups, as described below. The term “alkylamino” specifically refers to an alkyl group that is substituted with one or more amino groups, as described below, and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” a particular substituted alkynyl can be, e.g., an “alkynylsilyl,” a particular substituted aryl can be, e.g., a “nitroaryl,” a particular substituted cycloalkyl can be, e.g., a “cycloalkylether,” a particular substituted heterocycloalkyl can be, e.g., a “heterocycloalkylnitro,” a particular substituted cycloalkenyl can be, e.g., a “alkylcycloalkenyl,” a particular substituted heterocycloalkenyl can be, e.g., a “heterocycloalkenylthiol,” and the like.

The term “alkoxy” as used herein is an alkyl group bound through a single, terminal ether linkage; that is, an “alkoxy” group may be defined as —OA where A is alkyl as defined above.

The term “alkenyl” as used herein is a hydrocarbon group of from 2 to 24 carbon atoms with a structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxamate, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxamate, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxamate, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxamate, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and contains at least one double bound, e.g., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxamate, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H.

The terms “amine” or “amino” as used herein are represented by the formula NAA¹A², where A, A¹, and A² can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH.

The term “ester” as used herein is represented by the formula —OC(O)A or —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula AC(O)A¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxamate” as used herein is represented by the formula —C(O)NHOH.

The term “hydroxyl” as used herein is represented by the formula —OH.

The term “nitro” as used herein is represented by the formula —NO₂.

The term “silyl” as used herein is represented by the formula —SiAA¹A², where A, A¹, and A² can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfo-oxo” as used herein is represented by the formulas —S(O)A, —S(O)₂A, —OS(O)₂A, or —OS(O)₂OA, where A can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂A, where A can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “sulfone” as used herein is represented by the formula AS(O)₂A¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “sulfoxide” as used herein is represented by the formula AS(O)A¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

“Cy,” “R¹”, “R²,” and “L” as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is(are) selected will determine if the first group is embedded or attached to the second group.

Also described herein are the pharmaceutically acceptable salts and esters of compounds represented by Formula I (described below). By “pharmaceutically acceptable” is meant a material that is not biologically or otherwise undesirable, i.e., the material can be administered to an individual along with the selected compound without causing any undesirable biological effects or interacting in a deleterious manner with any of the other components of the pharmaceutical composition in which it is contained. Pharmaceutically acceptable salts can be prepared, for example, by treating the compound with an appropriate amount of a pharmaceutically acceptable base. Representative pharmaceutically acceptable bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. See for example, S. M. Berge, et al., “Pharmaceutical Salts,” J. Pharm. Sci., 66:1-19, 1977, which is incorporated herein by reference for its teaching of pharmaceutically acceptable salts. In one aspect, the reaction is conducted in water, alone or in combination with an inert, water-miscible organic solvent, at a temperature of from about 0° C. to about 100° C., such as at room temperature. The molar ratio of compounds represented by Formula I to be used is chosen to provide the ratio desired for any particular salts. For preparing, for example, the ammonium salt of a compound represented by Formula I, the compound can be treated with approximately one equivalent of a pharmaceutically acceptable base to yield a neutral salt. Pharmaceutically acceptable esters include, but are not limited to, methyl, ethyl, propyl, butyl, pentyl, hexyl, cyclopropyl, cyclobutyl, cyclopentyl, cyclohexyl, cycloheptyl, cyclopentenyl, cyclopentadienyl, cyclohexenyl, cyclohexadienyl, phenyl, pyridinyl, benzyl, and the like. Pharmaceutically acceptable esters can be prepared by, for example, by treating the compound with an appropriate amount of carboxylic acid, ester, acid chloride, acid anhydride, or mixed anhydride agent that will provide the corresponding pharmaceutically acceptable ester. Typical agents that can be used to prepare pharmaceutically acceptable esters include, for example, acetic acid, acetic anhydride, acetyl chloride, benzylhalide, benzaldehyde, benzoylchloride, methyl ethylanhydride, methyl phenylanhydride, methyl iodide, and the like.

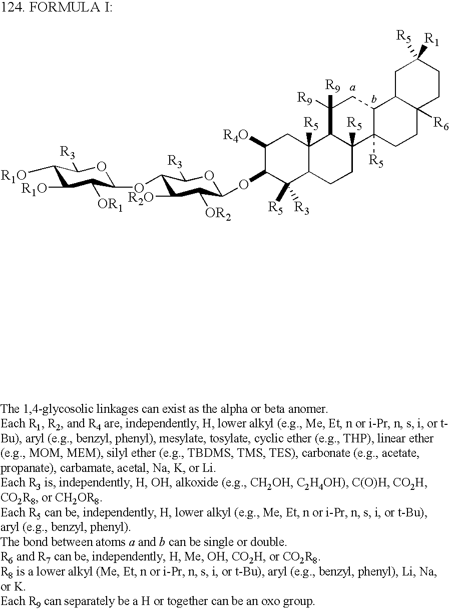

Examples of compounds, adjuvants, and derivatives of saponins can be found, for example, in U.S. Pat. Nos. 6,528,058, 6,645,495, 6,753,414, 6,524,584, 6,231,859, 5,688,772, 5,597,807, and 5,057,540. Each is herein specifically incorporated by reference for their teaching in regard to saponins and derivatives thereof which are useful with the methods disclosed herein.

For example, the saccharide side chains on the EsA molecule allows for high water solubility. The side chains can also improve the binding of the agent to the surface of targeted cells. After entering cells, the saccharide side chains are passively and/or enzymatically removed. The ring portion is the functional part of the agent.

Reference will now be made in detail to specific aspects of the disclosed materials, compounds, compositions, components, and methods, examples of which are illustrated in the following description and examples.

Unless stated to the contrary, a formula with chemical bonds shown only as solid lines and not as wedges or dashed lines contemplates each possible isomer, e.g., each enantiomer and diastereomer, and a mixture of isomers, such as a racemic mixture.

In one aspect, described herein are compositions comprising a compound represented by Formula I. In another aspect, described herein are compositions prepared by or with compounds represented by Formula I. For example, compounds represented by Formula I can be used as monomers in peptide synthesis. The use of amino acid monomers to synthesize peptides is well known in the art. Techniques for generating peptides from various amino acids, like those represented by Formula I, can involve solution based chemistry or solid phase chemistry, and can be performed on automated peptide synthesizers. Reviews of peptide syntheses that can be used to prepare peptides from the compounds disclosed herein can be found in Angew. Chem. Int. Ed. Engl., 24:799, 1985; Acc. Chem. Res., 22:47, 1989; Angew. Chem. Int. Ed. Engl., 30:1437, 1991; Pure & Appl. Chem., 59:331, 1987; Synthesis, 453, 1972; Angew. Chem. Int. Ed. Engl. 24:719, 1985, which are incorporated herein by reference for their teachings of peptide synthetic techniques. Disclosed herein are peptides comprising at least one compound represented by Formula I.

Compounds represented by Formula I can be optically active or racemic. The stereochemistry at the tertiary carbon shown in Formula I can vary and will depend upon the spatial relationship between the substituents on that carbon. In one aspect, the stereochemistry at the tertiary carbon shown in Formula I is S. In another aspect, the stereochemistry at the tertiary carbon shown in Formula I is R.

Using techniques known in the art, it is possible to vary the stereochemistry at the tertiary carbon shown in Formula I. While such enantioselective and enantiospecific techniques typically provide the one isomer, the presence of a minor amount of the other isomer can sometimes occur. As such, in one aspect, the compound represented by Formula I is the substantially pure S enantiomer. Alternatively, the compound represented by Formula I is the substantially pure R enantiomer. Also, depending on the particular R¹ and/or L group, other carbon stereocenters can exist in compounds represented by Formula I. The S and R isomers of such additional stereocenters are contemplated herein. Accordingly, Formula I includes enantiomers, diastereomers, and meso forms of the compounds represented thereby.

Compounds represented by Formula I can be readily synthesized using techniques generally known to those of skill in the art. The starting materials and reagents used in preparing these compounds are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4^{th} Edition); and Larock's Comprehensive Organic Transformations (VCH Publishers Inc., 1989).

While the synthetic routes discussed above can be performed as solution-phase multiple parallel syntheses, which involves the synthesis of compounds in individual reaction vessels, other methods can be performed. For example, combinatorial based syntheses or solid phase syntheses can be used and will depend on the particular compounds to be synthesized, the availability of reagents, or preference.

The compositions of the invention can be administered in vivo in a pharmaceutically acceptable carrier. By “pharmaceutically acceptable” is meant a material that is not biologically or otherwise undesirable, i.e., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Disclosed are compositions comprising EsA or a derivative thereof and a pharmaceutical carrier. Pharmaceutical carriers are known to those skilled in the art. These most typically would be standard carriers for administration of drugs to humans, including solutions such as sterile water, saline, and buffered solutions at physiological pH. The compositions can be administered in a number of ways, as described below. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including opthamalically, vaginally, rectally, intranasally), orally, by inhalation, or parenterally, for example by intravenous drip, subcutaneous, intraperitoneal or intramuscular injection. The disclosed compositions can be administered intravenously, intraperitoneally, intramuscularly, intraarticularly, intrathecally, subcutaneously, intracavity, or transdermally. The pharmaceutical compositions can also be administered in the form of an intraoperative wash.

The compositions disclosed herein can also be administered through topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization. The latter may be effective when a large number of animals are to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-trialkyl and aryl amines and substituted ethanolamines.

The dosage ranges for the administration of the compositions are those large enough to produce the desired effect of the methods disclosed herein. The dosage should not be so large as to cause adverse side effects, such as unwanted cross-reactions, anaphylactic reactions, and the like. Generally, the dosage will vary with the age, condition, sex and can be determined by one of skill in the art. The dosage can be adjusted by the individual physician in the event of any contraindications. Dosage can vary, and can be administered in one or more dose administrations daily, for one or several days. While individual needs vary, determination of optimal ranges of effective amounts of the vector is within the skill of the art. Typical dosages comprise about 0.01 to about 100 mg/kg-body wt. The preferred dosages comprise about 0.1 to about 100 mg/kg·body wt. The most preferred dosages comprise about 1 to about 100 mg/kg·body wt.

For example, 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, or 100 mg/kg or any amount in between of EsA or a derivative thereof can be administered to a subject for treatment of inflammation, pain, brain edema, angiogenesis, and COX-2 inhibition, for example. In one embodiment, EsA is administered in the amount of 2-40 mg/kg. In another embodiment, EsA is administered in the amount of 5-30 mg/kg. In another embodiment, EsA is administered in the amount of 5-20 mg/kg. An appropriate amount can be determined by one of ordinary skill in the art using only routine experimentation given the teachings herein.

Dosages can be given every 1, 2, 3, 4, 5, 6, 7, 8, 9, 10, 11, 12, 13, 14, 15, 16, 17, 18, 19, 20, 21, 22, 23, 24, 36, 48, or 72 hours, or any amount in between. It can also be given weekly, biweekly, monthly, or yearly, depending on the condition being treated and the individual needs of the subject receiving treatment. Dosages can also be administered in the form of a bolus. Dosages can also be administered preventatively in an effective amount that can be determined by one of ordinary skill in the art.

The materials may be in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, Bioconjugate Chem., 2:447-451, (1991); Bagshawe, K. D., Br. J. Cancer, 60:275-281, (1989); Bagshawe, Br. J. Cancer, 58:700-703, (1988); Senter, Bioconjugate Chem., 4:3-9, (1993); Battelli, Cancer Immunol. Immunother., 35:421-425, (1992); Pietersz and McKenzie, Immunolog. Reviews, 129:57-80, (1992); and Roffler, Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of cells in vivo. The following references are examples of the use of this technology to target specific proteins to tumor tissue (Hughes, Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release. This liposome delivery system can also be made to target B cells by incorporating into the liposome structure a ligand having an affinity for B cell-specific receptors.

Compositions including the liposomes in a pharmaceutically acceptable carrier are also contemplated.

Transdermal delivery devices have been employed for delivery of low molecular weight compositions by using lipid-based compositions (i.e., in the form of a patch) in combination with sonophoresis. However, as reported in U.S. Pat. No. 6,041,253 to Ellinwood, Jr. et al., which is hereby incorporated by reference in its entirety, transdermal delivery can be further enhanced by the application of an electric field, for example, by iontophoresis or electroporation. Using low frequency ultrasound which induces cavitation of the lipid layers of the stratum corneum, higher transdermal fluxes, rapid control of transdermal fluxes, and drug delivery at lower ultrasound intensities can be achieved. Still further enhancement can be obtained using a combination of chemical enhancers and/or magnetic field along with the electric field and ultrasound.

Implantable or injectable protein depot compositions can also be employed, providing long-term delivery of, e.g., EsA or derivatives thereof. For example, U.S. Pat. No. 6,331,311 to Brodbeck, which is hereby incorporated by reference in its entirety, reports an injectable depot gel composition which includes a biocompatible polymer, a solvent that dissolves the polymer and forms a viscous gel, and an emulsifying agent in the form of a dispersed droplet phase in the viscous gel. Upon injection, such a gel composition can provide a relatively continuous rate of dispersion of the agent to be delivered, thereby avoiding an initial burst of the agent to be delivered.

Disclosed herein are kits that can be used in practicing the methods disclosed herein. For example, a kit can comprise EsA or a derivative thereof. The kit can further comprise instructions, and a water soluble COX-2 inhibitor, such as EsA or a derivative thereof. The kits can include any reagent or combination of reagent discussed herein or that would be understood to be required or beneficial in the practice of the disclosed methods.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### A. EXAMPLES

### 1. Example 1

### IR-Induced Toxicity

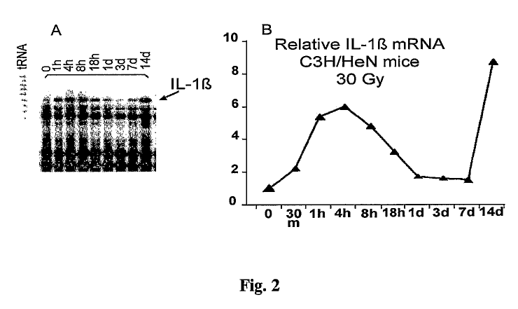

Alterations of IL1β and COX-2 in irradiated normal tissues. Using IR animal models, the key factors in soft tissue and brain IR toxicity have been identified, which can serve as targets for radioprotectors. IL1β is a key player in soft tissue IR toxicity. After screening several panels of cytokines, chemokines and lymphokines with RiboQuant™ Multi-Probe RNase Protection Assay System (PharMingen Co, San Diego, Calif.), IL1β levels were altered significantly as compared to other factors tested. To determine the timing of its change, a dynamic study was performed with different strains (C3H/HeN, Balb/c and C57BL/6) of mice. The mRNA levels of the skin of mice at various times following radiation were measured with RNase protection assay (FIG. 2A) and quantified by phosphorimaging (FIG. 2B). The results showed that the increased IL1β started at 1 hour after 30Gy IR, peaked at 4 hour and returned to almost background on day 3. Similar results were obtained from three strains of mice, indicating that this pattern is a common phenomenon. The time course of alteration demonstrated when to use the radioprotector at the first wave of IR reaction. To achieve a protective effect on early stage, EsA can be administrated a few hours before exposure to IR.

The second wave of IL1β surged on day 14 with a level much higher than that in the early acute phase (FIG. 2). The underlying mechanism of this phenomenon is an immune-like response. The IR induced free radicals that damage the tissues, which causes the production and release of IL1β and other acute-response factors that attract both unspecific host immune cells (such as macrophages and neutrophils) and specific immune cells (such as T and B lymphocytes) to the IR location. The interaction of macrophages (antigen presenting cells) with lymphocytes lays down the cellular foundation of the IR toxicity that normally occurs weeks and months after a single dose or course of radiation. It is these immune cells that recognize the damage and cause the second and subsequent waves of IL1β. These data show that the relative long-term use (>2-3 weeks after IR) of EsA blocked the second wave of IL1β and prevented the early IR skin toxicity.

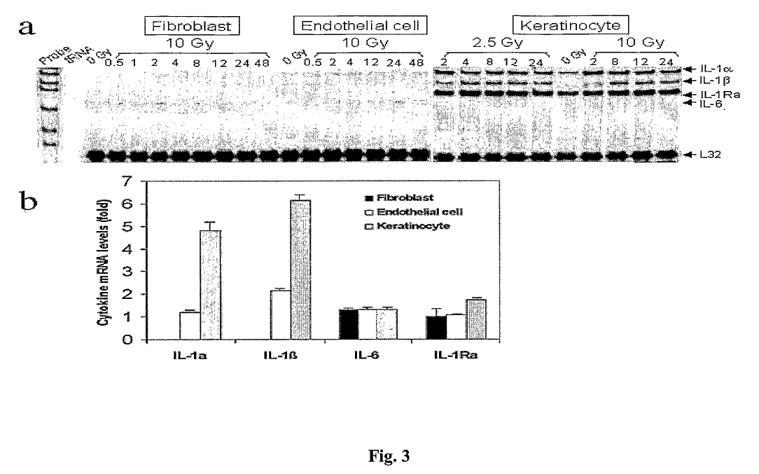

IL1β related skin damage is an overall result of changes taking place in all cell components of skin. Primary human keratinocytes, vascular endothelium and fibroblasts (Clontech, Inc, CA) were cultured and exposed to 2.5 to 10 Gy IR. The results (FIGS. 3A and B) show that the irradiated keratinocytes are the major producers of IL1. Therefore, when testing the effect of EsA, the keratinocytes is a good target cell type and their IL1 production level is a good index for inhibition efficiency. IL-6 was not affected, thus the radiation response is a relatively specific inflammatory reaction prominently involving the IL-1 signal pathway. IL-1 is a good molecular target for prevention of radiation toxicity. Since the keratinocytes are on the surface of skin, EsA can be formulated as a cream to reduce the IL-1 from keratinocytes.

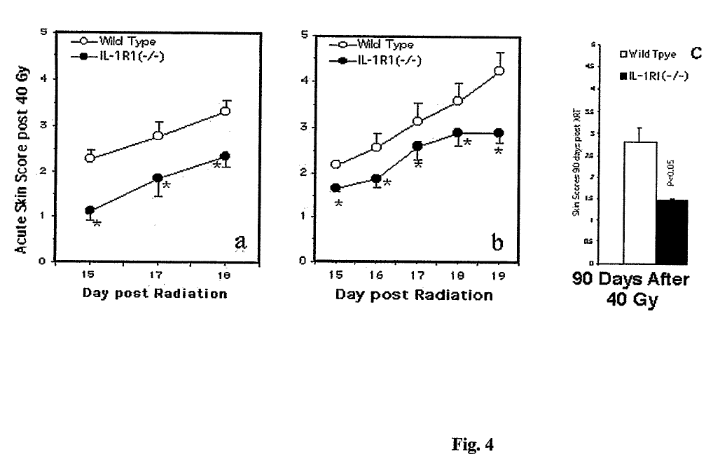

To further confirm that IL1β plays a key role in IR skin toxicity, the hind limbs of IL1 R1−/− mice were irradiated with a single dose of 40 Gy and skin damage was observed. Skin scores were measured at both the early phase, which reaches a peak at 18 to 20 days and at late time points (90 days). The results show that IL-1R1−/− mice consistently have lower tissue damage as compared to their wild type counterparts (C57BL/6) (FIGS. 4A and B, * indicates p<0.05). The loss of IL-1R1 receptor lead to no signal transduction mediator of IL1β, and therefore, blocked the IL1β signaling path and reduces the degree of IR skin damage. The incomplete blocking indicated the existing of other inflammatory mediators, which can also be the targets. This set of data demonstrated that IL1β is a major player in IR skin damage and a good target for radioprotectors, such as EsA.

### 2. Example 2

### IL1β and COX-2 is the Key Players in Brain IR Toxicity

The data in the screening of panels of the different biological factors at mRNA levels in irradiated mouse brain demonstrated that IL1β and COX-2 were major responders upon 35 Gy IR (Table 1). The up-regulation of IL1β was in a dose-dependent and time-dependent manner (Table 2 and 3).

TABLE 1
Changes of mRNA of IL1β and
COX2 in mouse brain upon 35 Gy IR

| | 0 Gy | 35 Gy |
|---|---|---|
| IL1β | 1 | 3.75* |
| COX2 | 1 | 3.78* |

(fold increase)

TABLE 2
Dose-Dependent Changes of mRNA of IL1β in
mouse brain upon 35 Gy IR

| | 0 Gy | 5 Gy | 15 Gy | 25 Gy | 35 Gy |
|---|---|---|---|---|---|
| IL1β | 1 ± 0.2 | 1.5 ± 0.2 | 14 ± 3* | 20 ± 3* | 23 ± 3* |

(fold increase, mean ± SD, n = 5)

TABLE 3
Time-Dependent Changes of mRNA of IL1β in
mouse brain upon 35 Gy IR

| | 0 hour | 4 hour | 24 hour |
|---|---|---|---|
| IL1β | 1 ± 0.2 | 2.5 ± 0.2* | 3.1 ± 0.3* |

(fold increase, mean ± SD, n = 5)

When alteration of mRNA of IL1β and IL1α were simultaneously determined, the mRNA of IL1β increased about 6-fold while mRNA of IL1α remained unchanged, demonstrating that IL1β is the main player in IR induced toxicity. In other models IL1α can also be a major factor mediating the IL1 signal. The role of COX-2 in acute IR brain toxicity (mainly brain edema) was further confirmed in an experiment in which a selective COX-2 inhibitor, NS-398, blocked the induction of prostanoid and the IR brain edema (Moore et al. Radiat Res. 161(2):153-60 (2004). These data demonstrate that IL1β and COX-2 are targets for the therapeutical intervention of brain edema.

### 3. Example 3

### EsA Protect Normal Tissues from IR-Toxicity In Vivo

The effect of EsA was tested on protection of early IR toxicity in both soft tissue (skin) and brain models.

TABLE 4
Preclinical Criteria for IR-induced early Toxicity in Soft Tissue

| | |
|---|---|
| 1.0 | Normal |
| 1.5 | Slight erythema |
| 2.0 | Depigmentation with <25% hair loss |
| 2.5 | Early dry desquamation, thickening, >25% hair loss |
| 3.0 | Dry desquamation, mild edema, |
| 3.5 | Dry desquamation, early moist |
| 4.0 | Moist desquamation, moderate <50% |
| 4.5 | >50% desquamation ± some necrosis |
| 5.0 | Significant necrosis and loss of dermis |

(<2 months, peaked at 3-4 weeks)

EsA protects the soft tissue from IR-induced damage. IR sensitive and IL1β high expressing C57BL/6 mice are irradiated with 30 Gy and the skin damage is measured with a preclinical criteria (Table 4).

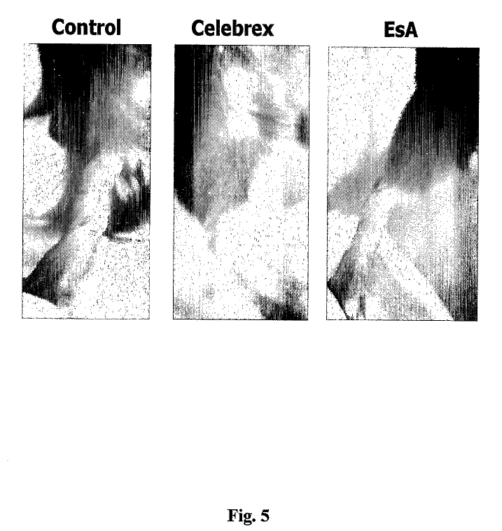

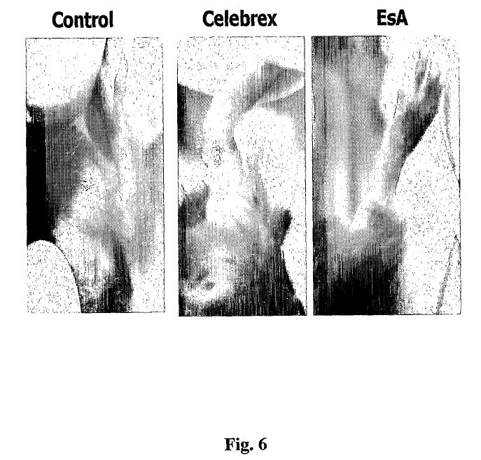

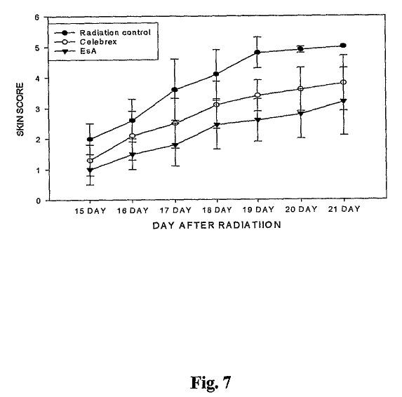

The effect of EsA was examined with this model system. The mice (5/group) were i.p. injected with 10 mg/kg EsA (as test) or PBS vehicle (as control) or intragastrical administration of 50 mg/kg Celebrex (as positive drug control) 16 hour before and then daily after 30 Gy single dose IR for 4 weeks. The results showed that on day 19, there was a significant difference in the degree of skin damage. While the control mice had a moist desquamation (score above 4.5), the EsA treated mice had only erythema (score about 2) and Celebrex had a score of about 3 (FIG. 5). 4 weeks after IR, Celebrex lost its protection effect while EsA effectively protected the soft tissue (FIG. 6). The difference was statistically significant (FIG. 7).

A repeat experiment was performed with an increased number of mice (10 mice/group), and the EsA protective effect was confirmed. During the whole course of EsA treatment, no signs of sickness were observed in mice treated with EsA. In addition, at the end of the experiments, mice treated with EsA had body weights similar to the vehicle control group, indicating that EsA is safe. The data show that this traditional anti-inflammation agent is a radiation modulator for soft tissue damage.

### 4. Example 4

### EsA Protects the Brain from IR-Induced Edema

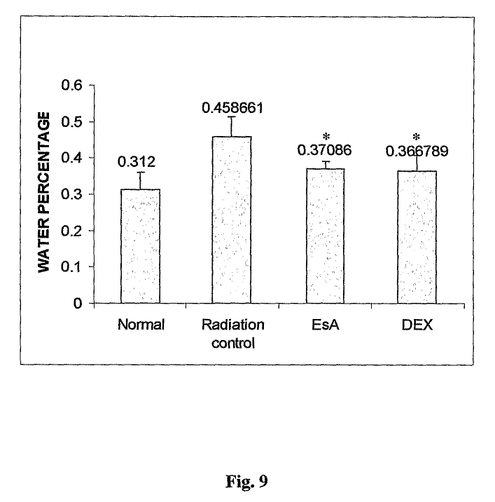

EsA has a strong inhibitory effect on erythema and can reduce IR-induced brain edema. For this, the mice (5/group) were i.p. injected with 10 mg/kg EsA or PBS vehicle or i.v. 3 mg/kg Dexamethasome (Dex, as positive drug control) 18 hour before the entire heads of the mice (5/group) were irradiated at 40 Gy single dose. The mice without radiation served as negative controls. Twenty four hours later, the whole brain was taken out, weighed (wet weight), placed on aluminum foil and placed in an oven at 60° C. At different time points (10 min intervals for the first 2 hours and then 4, 6, 24 hr), the brains were weighed, and the results were expressed as: % water={(wet weight−dry weight)/wet weight}×100%. The higher the % water, the greater the severity of edema. Brain % water as a function of time provided a brain drying curve. This method allowed for observation of brain edema in a more detailed way, since it better detects water that is drying from different microanatomic tissue compartments. IN the case of edema, a larger portion of water leaks out from damaged vessels into interstitial space and dries faster than water trapped in cells. Dynamic measurement of the water evaporation provides this information. The results (FIG. 8) showed that the brains of irradiated mice without EsA had a higher edema water percentage than those treated with EsA or Dex. The reduction of brain edema was statistically significant (from first hour data, P<0.05, FIG. 9).

A whole-body MR scanner can be used to accurately measure the degree of edema. Using a circular coil of diameter 2.2 cm, images of mouse brain were obtained at the resolution of 150×150×160 microns in 5 minutes. A more advanced whole-body 3.0 Tesla (T) MR scanner was used with a dual phased array RF receiver coils composed of two orthogonal surface coil elements about 2 cm in diameter to achieve resolution of 80×80×160 microns. This type of coil design has obtained a high-resolution imaging in the wrist (Kwok et al. Magn Reson Med. 43(3):335-41 (2000 March;) and the mice (Totterman et al. AJR 156:343-344 (1991). Combined with three-dimensional double echo gradient echo sequence and scans of 32 slices covering the entire brain, brain water content was determined in a highly sensitive and precise way.

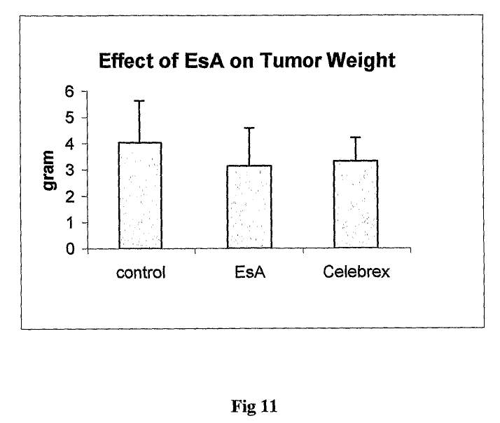

Considering that EsA is used as a radioprotector in patients with tumors, its effect on tumor growth is a critical issue for its application. For this, Lewis' lung carcinoma cells were inoculated in syngeneic C57BL/6 mice and treated with or without EsA or Celebrex at the same dose used in sort tissue protection daily for 20 days. The results (FIG. 11) indicated that EsA had little effect on tumor growth, i.e., neither stimulation nor inhibition of tumor growth, demonstrating that it is safe for use in cancer patients for protecting normal tissue while not promoting tumor growth.

### 5. Example 5

### EsA does not Possess Activity of Steroidal Hormones

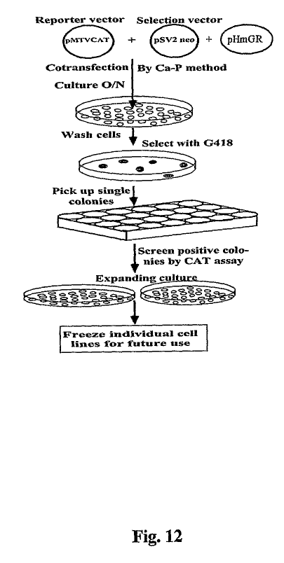

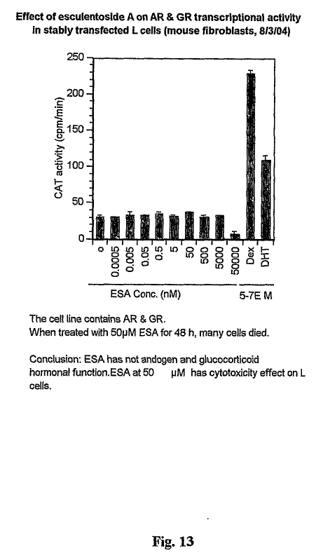

Structurally, EsA has a certain similarity with steroidal hormone, especially a steroid-like back-bone. Functionally, it exerts anti-brain edema effects, which are clinically obtained with Dexamethasome. A sensitive reporter system for the hormone transactivity of glucocorticoids receptor (GR) and androgen receptor (AR) was set up as elucidated in the chart (FIG. 12). E8.2.A3 cells derived from L cells (mouse fibroblasts, 41) lack GR, but contain high levels of AR. The cells were cotransfected with wild type mouse GR expression vector (pmGR), reporter vector pMTVCAT and a selection vector pSV2neo vector at a ratio of 30 μg:5 μg:0.5 μg in 100 mm dish using the calcium phosphate precipitation method. Individual clones with stably transfected mGR and CAT reporter gene were selected with 400 μg/ml of G418 in DMEM medium containing 3% charcoal stripped new born calf serum. Then, the cells were seeded (5×10⁵/well) in 24 well plates and treated without (as negative control) or with 5×10⁻⁷ M of Dexamethasome (Dex) and dihydrotestosteron (DHT) as positive control or with different concentrations of ESA (as test) in triplicate for 44 hours. The cells were observed for morphological changes twice a day. There is no change below 4 mg/ml and only at the level of 40 mg/ml was death of cells observed. Upon interaction with steroid hormone (if any), the existing GR and AR are activated and the transactivational activity can be detected by the CAT assay (Zhang et al. Mol Endocrinol 10(1): 24-34 (1996)). The treated cells were washed with phosphate buffer saline (PBS) once. To each well, 0.25 ml of 0.25M Tris-HCl was added. The plates underwent 3 cycles of freeze and thaw at −70° C. and room temperature followed by heating at 65° C. for 10 min by floating the plates in a hot water bath. Then, 0.1 ml of cell lysate from each well was used for the CAT assay. The assay was performed as described previously (Zhang et al. (1996)). Briefly, 100 μl of cell lysate from each well were mixed with 150 μl reaction solution (128 μl of 0.25 M Tris-HCl, pH 7.8, 2 μl of the commercial ³H-acetyl CoA and 20 μl of 2 mg/ml chloramphenicol) in the scintillation vials. To each vial, 2 ml non-aqueous scintillation fluid was added and mixed with the reaction by shaking or vortexing. The vials were counted 3-6 times in a scintillation counter at regular intervals over 1 to 4 hours. CAT activity was expressed in the rate of the reaction calculated by subtracting total cpm of one counting from total cpm of its previous counting and then dividing by time (min) between these two counts. This CAT activity represents transcriptional activities of the GR and AR, depending on what steroid hormone is used to induce CAT expression. The results show that Dex and DHT, as positive controls, had a high level of transactivity as expected., indicating that the test system is working well. However, the EsA at all the test concentrations (from 0, 0.4, 4, 40, 400 ng to 4, 40, 400, 4000, 40000 μg/ml) had neither androgen nor glucocorticoidal hormone transactivity (FIG. 13), indicating that the EsA is a non-steroidal substance. This is a very important feature, since few nonsteroidal anti-inflammatory drugs (NSAIDs) exert anti-IR induced brain edema or skin damage.

### 6. Example 6

### EsA Reduces the NO Production

The anti-tumor effectiveness of IR is based in part on triggering the reactive oxygen species and free radicals in tumors; meanwhile, IR also damages normal tissues and causes unwanted toxicity. The IR toxicity can be reduced by superoxide dismutase gene therapy (Epperly et al. Int J Radiat Oncol Biol Phys. 26(3): 417-25 (1993 June). EsA's protective effects are mediated at least in part by down regulation of free radical production as demonstrated by the examination of the NO production in Raw264.7, a mouse macrophage cell line that had been irradiated. The assay was carried out based on the principle: in aqueous solution, nitric oxide rapidly degrades to nitrate and nitrite. The nitrite is a stable product and its accumulation represents the amount of NO. To accurately measure the NO a colorimetric Assay kit for NO (Oxford Product # NB 88) was used, in which the affinity purified nitrate reductase was used to convert nitrate to nitrite that was then quantitated with Griess Reagent. The linearized standard curve indicated that the assay was functional.

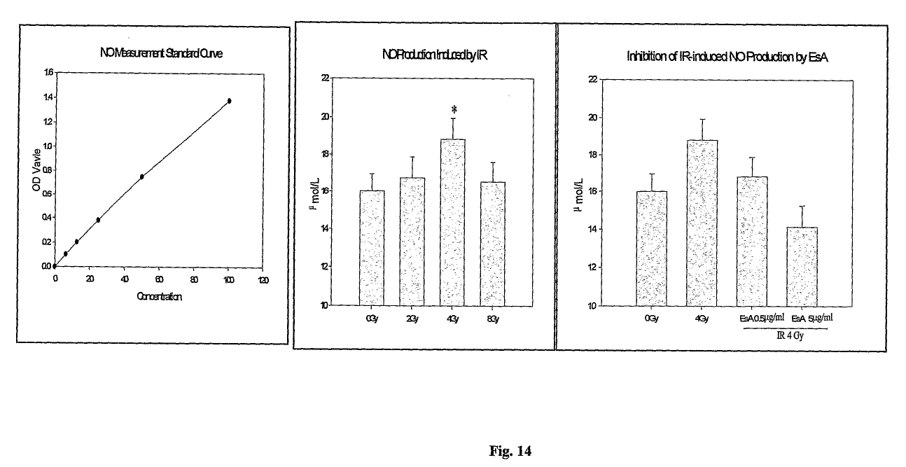

The study was carried out in Raw264.7 cells that were irradiated with 0, 2, 4 and 8 Gy. The dose of 4 Gy was found to have the best production of NO (FIG. 14B). At this optimal condition, EsA (0.5 or 5 μg/ml) was added 8 hours before the IR at 4 Gy and 24 hours later, the media was harvested and 100 μl of media was measured for NO content in the form of nitrite. The results show that the EsA inhibited the IR-induced NO production (FIG. 14C). To see if this inhibition was cell line specific, another human macrophage cell line THP-1 was examined. While the production of NO was greatly up-regulated (5 fold) 4 hours after IR, it was reduced (3 fold) upon the treatment with 0.1, 0.5 and 5 μg/ml EsA.

### 7. Example 7

### EsA Inhibits COX-2 Activity

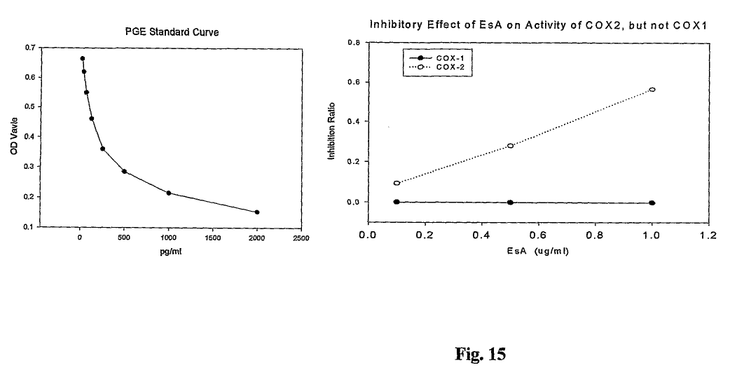

IR toxicity in brain and soft tissue was protected by other anti-COX-2 agents, such as Celebrex and NS-398 (a selective COX-2 inhibitor). EsA also possesses anti-COX-2 activity. To demonstrate this, a sophisticated system for screening COX inhibitors was used. It is known that constitutive COX-1 and inducible COX-2 catalyze the production of prostaglandins (PGs) from arachidonic acid. The Cayman COX Inhibitor Screening Assay kit directly measures PGF2α produced by SnCl2 reduction of COX-derived PGH2. The prostanoid product is quantified via enzyme immunoassay (EIA) using a broadly specific antibody that binds to all the major prostaglandin compounds (FIG. 15A). To distinguish the inhibition of COX-1 from COX-2, both ovine COX-1 and human recombinant COX-2 enzymes were used as targets. The EsA was applied to this specific testing system and the results (FIG. 15B) demonstrated that EsA had no effect on COX-1, but inhibited the COX-2 in a dose-dependent manner. EsA is a novel COX-2 inhibitor, which accounts for its observed protective effects on skin (FIGS. 5-7) and brain (FIGS. 8-9).

### 8. Example 8

### EsA Reduces IR-Induced IL1β Production

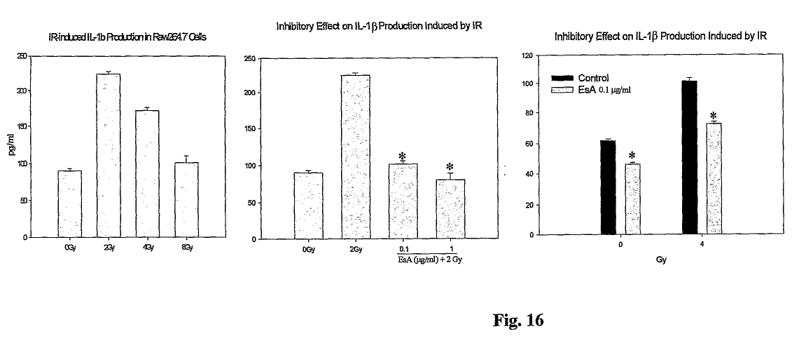

IL1β is crucial mediator in early IR toxicity of both brain and soft tissue. EsA has a potent inhibitory effect on LPS-induced IL1 production. The protective effect of EsA on early IR toxicity results in part from this effect. First we determined the optimal IR dose to induce IL1 production in Raw 264.7 macrophage cells. Equal numbers of cells were exposed to 0, 2, 4 and 8 Gy IR and 24 hours later, the conditioned media were harvested and 100 μl of media from each sample was measured for IL1β concentration by specific IL1β ELISA using monoclonal antibodies (purchased from R&D Systems, Inc). The data (FIG. 16A) show that IL1β was indeed induced by IR insult with a maximum induction at 2 Gy. Therefore, 2 Gy was used as optimal IR dose for Raw 264.7 for this set of tests. The cells were treated with 0.1 or 1 μg/ml of EsA for 18 hours, irradiated at 2Gy and then cultured for another 24 hours. The media were measured for protein level of IL1β by ELISA. The results (FIG. 16B) show that EsA at a dose of 0.1 μg/ml dramatically inhibited IR-induced production of IL1β. At 1 μg/ml, it reduced the IL1β to the basal level, indicating that the EsA is able to completely block the IR-induced IL1β production in this cell line (P<0.01). In another set of experiments, the IL1β induced by 4Gy IR in A431 human epidermoid carcinoma cells are inhibited by 0.1 μg/ml EsA (P<0.01, FIG. 16C), indicating that EsA acts on many cell types and can universally exerts its effect on certain molecules/pathways.

Throughout this application, various publications are referenced. The disclosures of these publications in their entireties are hereby incorporated by reference into this application in order to more fully describe the state of the art to which this invention pertains.

### REFERENCES

Abal M, Andreu J M, Barasoain I. Taxanes: microtubule and centrosome targets, and cell cycle dependent mechanisms of action. Curr Cancer Drug Targets. 2003 June; 3(3):193-203
Achiwa, H., Yatabe, Y., Hida, T., Kuroishi, T., Kozaki, K., Nakamura, S., Ogawa, M., Sugiura, T., Mitsudomi, T., Takahashi, T. (1999) Prognostic significance of elevated cyclooxygenase 2 expression in primary, resected lung adenocarcinomas. Clin. Cancer Res. 5, 1001-1005
Alisauskas R M, Goldenberg D M, Sharkey R M, Blumenthal R D. Reduction in the duration of myelotoxicity associated with radioimmunotherapy with infusions of the hemoregulatory peptide, HP5b in mice. Int J. Cancer. 1997 Jan. 27; 70(3):323-9.
Arun B, Goss P. The role of COX-2 inhibition in breast cancer treatment and prevention. Semin Oncol. 2004 April; 31(2 Suppl 7):22-9
Asanuma T, Fujihara T, Otani K, Miki A, Ishikura F, Beppu S, Noninvasive vessel-selective perfusion imaging with intravenous myocardial contrast echocardiography. J Am Soc Echocardiogr. 2004; 17(6): 654-8.
B. M. Fenton, S. F. Paoni, B. K. Beauchamp, and I. Ding, Zonal image analysis of tumour vascular perfusion, hypoxia, and necrosis, Br J Cancer 2002; 86:1831-1836.
B. M. Fenton, S. F. Paoni, J. Lee, C. J. Koch, and E. M. Lord, Quantification of tumor vascular development and hypoxia by immunohistochemical staining and HbO₂ saturation measurements, British Journal of Cancer 1999; 79:464-471.
Bamba, H., Ota, S., Kato, A., Adachi, A., Itoyama, S., Matsuzaki, F. (1999) High expression of cyclooxygenase-2 in macrophages of human colonic adenoma. Int. J. Cancer 83, 470-475
Barr, R. M, Walker, S. L, Tsang, W, Harrison, G. I, Ettehadi, P, Greaves, M. W, and Young, A. R. Suppressed alloantigen presentation, increased TNF-alpha, IL-1, IL-1Ra, IL-10, and modulation of TNF-R in UV-irradiated human skin, J Invest Dermatol. 112: 692-8, 1999.
Ben-Av, P., Crofford, L. J., Wilder, R. L., Hla, T.: Induction of vascular endothelial growth factor expression in synovial fibroblasts by prostaglandin E and interleukin-1: a potential mechanism for inflammatory angiogenesis. FEBS Lett. 372, 83-87, 1995
Berenbaum M C, Hall G W, Hoyes A D. Cerebral photosensitization by hematoporphyrin derivative. Evidence for an endothelial site of action. Br J. Cancer. 1986; 53(1):81-9
Biswas P S, Banerjee K, Kim B, Rouse B T. Mice transgenic for IL-1 receptor antagonist protein are resistant to herpetic stromal keratitis: possible role for IL-1 in herpetic stromal keratitis pathogenesis. J Immunol. 2004 Mar. 15; 172(6):3736-44.
Bogo V, Jacobs A J, Weiss J F. Behavioral toxicity and efficacy of WR-2721 as a radioprotectant. Radiat Res. 1985 November; 104(2 Pt 1):182-90.
Bol D K, Rowley R B, Ho C P, Pilz B, Dell J, Swerdel M, Kiguchi K, Muga S, Klein R, Fischer S M. Cyclooxygenase-2 overexpression in the skin of transgenic mice results in suppression of tumor development. Cancer Res. 2002 May 1; 62(9):2516-21
Boquillon M, Boquillon J P, Bralet J. Photochemically induced, graded cerebral infarction in the mouse by laser irradiation evolution of brain edema. J Pharmacol Toxicol Methods. 1992; 27(1): 1-6.
Boutin H, Kimber I, Rothwell N J, Pinteaux E. The expanding interleukin-1 family and its receptors: do alternative IL-1 receptor/signaling pathways exist in the brain? Mol. Neurobiol. 2003; 27(3):239-48
Caivano, M., Cohen, P. (2000) Role of mitogen-activated protein kinase cascades in mediating lipopolysaccharide-stimulated induction of cyclooxygenase-2 and IL-1 beta in RAW264 macrophages. J. Immunol. 164, 3018-3025
Castelli M P, Black P L, Schneider M, Pennington R, Abe F, Talmadge J E Protective, restorative, and therapeutic properties of recombinant human IL-1 in rodent models. J Immunol. 1988 Jun. 1; 140(11):3830-7
Chang et al., “Role of prostaglandin E2-dependent angiogenic switch in cyclooxygenase-2-induced breast cancer progression,” PNAS, DOI:10.1073/pnas.2535911100, Dec. 15, 2003.
Chen J, Xu X, Underhill C B, Yang S, Wang L, Chen Y, Hong S, Creswell K, Zhang L: Tachyplesin Activates the Classical Complement Pathway to Kill Tumor Cells. Cancer Res. (accepted)
Chen, Y, Rubin, P, Williams, J, Hernady, E, Smudzin, T, and Okunieff, P. Circulating IL-6 as a predictor of radiation pneumonitis, Int J Radiat Oncol Biol Phys. 49: 641-8, 2001.
Chen, Y, Williams, J, Ding, I, Hernady, E, Liu, W. M, Smudzin, T, Finkelstein, J, and Okunieff, P. Radiation pneumonitis and early circulatory cytokine markers, Seminars in Radiation Oncology. 12: 26-33, 2002.
Cohen, E. P, Bonsib, S. A, Whitehouse, E, Hopewell, J. W, and Robbins, M. E. Mediators and mechanisms of radiation nephropathy, Proc Soc Exp Biol Med. 223: 218-25, 2000.
Crofford, L. J., Tan, B., McCarthy, C. J., Hla, T. (1997) Involvement of nuclear factor kappa B in the regulation of cyclooxygenase-2 expression by interleukin-1 in rheumatoid synoviocytes. Arthritis Rheum. 40, 226-236
Danielsson G, Norgren L, Truedsson L, Andreasson A, Danielsson P, Nilsson A, Swartbol P. Flavonoid treatment in patients with healed venous ulcer: flow cytometry analysis suggests increased CD11b expression on neutrophil granulocytes in the circulation. Vasc Med. 2003; 8(2):83-8.
DiPaola R S, Schuchter L. Neurologic protection by amifostine. Semin Oncol. 1999 April; 26(2 Suppl 7):82-8
Dubray, B, Delanian, S, and Lefaix, J. L. Late effects of mammary radiotherapy on skin and subcutaneous tissues, Cancer Radiother. 1: 744-52, 1997.
Emami B, Lyman J, Brown A, Coia L, Goitein M, Munzenrider J E, et al. Tolerance of normal tissue to therapeutic radiation. Int J Radiat Oncol Biol Phys 1991; 21:109-22.
Epperly M W, Travis E L, Sikora C, Greenberger J S. Manganese [correction of Magnesium] superoxide dismutase (MnSOD) plasmid/liposome pulmonary radioprotective gene therapy: modulation of irradiation-induced mRNA for IL-I, TNF-alpha, and TGF-beta correlates with delay of organizing alveolitis/fibrosis. Biol Blood Marrow Transplant. 1999; 5(4):204-14.
Fang J, Zheng Q Y. Inhibitory effect of esculentoside A on platelet activating factor released from calcimycin induced rat peritoneal macrophages. Yao Xue Xue Bao. 1991; 26(10):721-4.
Felemovicius I, Bonsack M E, Baptista M L, Delaney J P. Intestinal radioprotection by vitamin E (alpha-tocopherol). Ann Surg. 1995 October; 222(4):504-8; discussion 508-10.
Felies M, Von Horsten S, Pabst R, Nave H. Neuropeptide Y stabilizes body temperature and prevents hypotension in endotoxemic rats. J. Physiol. 2004 Sep. 23
Fiebich, B. L., Mueksch, B., Boehringer, M., Hull, M. (2000) Interleukin-1 beta induces cyclooxygenase-2 and prostaglandin E(2) synthesis in human neuroblastoma cells: involvement of p38 mitogen-activated protein kinase and nuclear factor-kappaB. J. Neurochem. 75, 2020-2028
Friedman E J. Immune modulation by ionizing radiation and its implications for cancer immunotherapy. Curr Pharm Des. 2002; 8(19):1765-80.
Fu S L, Wu Y L, Zhang Y P, Qiao M M, Chen Y. Anti-cancer effects of COX-2 inhibitors and their correlation with angiogenesis and invasion in gastric cancer. World J. Gastroenterol. 2004 Jul. 1; 10(13):1971-4.
Gabizon, A. Liposome circulation time and tumor targeting: Implications for cancer chemotherapy. Adv. Drug Delivery Rev. 1995; 16, 285-294.
Gardner R V. Interferon-gamma (IFN-gamma) as a potential radio- and chemo-protectant. Am J. Hematol. 1998 July; 58(3):218-23
Goitein, M. Future prospects in planning radiation therapy, Cancer. 55: 2234-9, 1985.
Hac S, Dobosz M, Kaczor J, Rzepko R. Influence of molecule CD 11b blockade on the course of acute ceruleine pancreatitis in rats. Exp Mol. Pathol. 2004; 77(1):57-65.
Hallahan D E, Haimovitz-Friedman A, Kufe D W, Fuks Z, Weichselbaum R R. The role of cytokines in radiation oncology. Important Adv Oncol. 1993:71-80.
Han Z, Ni J, Smits P, Underhill C B, Xie B, Chen Y, Liu N, Tylzanowski P, Parmelee D, Feng P, Ding I, Gao F, Gentz R, Huylebroeck D, Merregaert J, and Zhang L: Extracellular matrix protein 1(ECM1): a novel angiogenic factor expressed in breast cancer cells. FASEB J: 2001; 988
Hassink, E. A. M., Souren, T. S., Boersma, L. J., Peerboom, P. F., Melkert, R., van Zandwijk, N., Lebesque, J. V. and Bruning, P. F. Pulmonary morbidity 10-18 years after irradiation for Hodgkin's disease. Eur. J. Cancer, 1992; 29A, 343-347.
Heitjan D F, Manni A, Santen R J: Statistical analysis of in vivo tumor growth experiments. Cancer Res 1993; 53(24): 6042-50
Hensley M L, Schuchter L M, Lindley C, Meropol N J, Cohen G I, Broder G, Gradishar W J, Green D M, Langdon R J Jr, Mitchell R B, Negrin R, Szatrowski T P, Thigpen J T, Von Hoff D, Wasserman T H, Winer E P, Pfister D G. American Society of Clinical Oncology clinical practice guidelines for the use of chemotherapy and radiotherapy protectants. J Clin Oncol. 1999 October; 17(10):3333-55.
Herbst R S, Khuri F R. Mode of action of docetaxel—a basis for combination with novel anticancer agents. Cancer Treat Rev. 2003 October; 29(5):407-15
Hirsch, E., Irikura, V., Paul, S. M. and Hirsh, D. Functions of interleukin 1 receptor antagonist in gene knockout and overproducing mice. Proc. Natl. Acad. Sci. USA 1996 93, 11008-11013.
Housley, P. R.; Forsthoefel, A. M. Isolation and characterization of a mouse 1 cell variant deficient in glucocorticoid receptors. Biochem Biophys Res Commun 1989, 164(1), 480-487.
http://216.239.39.104/search?q=cache:BOxXCEAhTawJ:www.holliseden.com/resources/MMT-article.pdf+ionizing+radiation+and+protectant&hl=en
http://jaxmice.jax.org/jaxmice-cgi/jaxmicedb.cgi?objtype=pricedetail&stock=003018&dest=N
http://jaxmicetjax.org/jaxmice-cgi/jaxmicedb.cgi?objtype=pricedetail&stock=002609
http://journals.ons.org/xp6/ONS/Library.xml/ONS_Publications.xml/CJON.xml/CJON2001.xml/July_August_{—}2001.xml/Members_Only/Integrated_Care.xml
HTTP://www.ALZFORUM.ORG/RES/COM/TRA/COX/KELLEY.ASP
http://www.cancerpage.com/news/article.asp?id=4516
http://www.caymanchem.com/neptune/servlet/neptune/catalog/560131/a/z/template/Product.vm
http://www.dmd.nl/cav3_home.html
http://www.emdbiociences.com/product/70637
http://www.lef.org/magazine/mag2001/nov2001_report_scans_{—}02.html
http://www.oxfordbiomed.com/PDF-Kit %20Inserts/nb88r.pdf
http://www.taconic.com/anmodels/002181.htm
http://www.thorne.com/altmedrev/fulltext/glut.html
Ishida 0, Maruyama K, Tanahashi H, Iwatsuru M, Sasaki K, Eriguchi M, Yanagie H.: Liposomes bearing polyethyleneglycol-coupled transferrin with intracellular targeting property to the solid tumors in vivo. Pharm Res 2001; 18(7):1042-8
Iyama, K.; Zhang, S.; Lo, S. C. Effects of mycoplasmal lamps on receptor responses to steroid hormones in mammalian cells. Curr Microbiol 2001, 43(3), 163-169.
Jaeckel E C, Raja S, Tan J, Das S K, Dey S K, Girod D A, Tsue T T, Sanford T R. Correlation of expression of cyclooxygenase-2, vascular endothelial growth factor, and peroxisome proliferator-activated receptor delta with head and neck squamous cell carcinoma. Arch Otolaryngol Head Neck Surg. 2001 October; 127(10):1253-9.
Johansen, J, Bentzen, S. M, Overgaard, J, and Overgaard, M. Relationship between the in vitro radiosensitivity of skin fibroblasts and the expression of subcutaneous fibrosis, telangiectasia, and skin erythema after radiotherapy, Radiother Oncol. 40: 101-9, 1996.
Ju D W, Zheng Q Y, Cao X, Fang J, Wang H B. Esculentoside A inhibits tumor necrosis factor, interleukin-1, and interleukin-6 production induced by lipopolysaccharide in mice. Pharmacology. 1998 April; 56(4):187-95
Ju D W, Zheng Q Y, Wang H B, Guan X J, Fang J, Yi Y H.: Inhibitory effects of esculentoside A on mouse macrophages and antibody production. Yao Xue Xue Bao. 1994; 29(4):252-5
Kakiuchi, Y., Tsuji, S., Tsujii, M., Murata, H., Kawai, N., Yasumaru, M., Kimura, A., Komori, M., Irie, T., Miyoshi, E., et al: Cyclooxygenase-2 activity altered the cell-surface carbohydrate antigens on colon cancer cells and enhanced liver metastasis. Cancer Res. 62, 1567-1572, 2002
Kalechman Y, Shani A, Albeck M, Sredni B Induction of acute phase proteins in mice and humans by treatment with AS101, an immunomodulator with radioprotective properties. Immunopharmacology. 1995 March; 29(2): 149-58
Khan R B, Krasin M J, Kasow K, Leung W. Cyclooxygenase-2 inhibition to treat radiation-induced brain necrosis and edema. J Pediatr Hematol Oncol. 2004; 26(4):253-5.
Kirtikara, K., Raghow, R., Laulederkind, S. J., Goorha, S., Kanekura, T., Ballou, L. R. (2000) Transcriptional regulation of cyclooxygenase-2 in the human microvascular endothelial cell line, HMEC-1: control by the combinatorial actions of AP2, NF-IL-6 and CRE elements. Mol. Cell. Biochem. 203, 41-51
Kone B C. Protein-protein interactions controlling nitric oxide synthases. Acta Physiol Scand. 2000 January; 168(1):27-31
Kuwano T, Nakao S, Yamamoto H, Tsuneyoshi M, Yamamoto T, Kuwano M, Ono M. Cyclooxygenase 2 is a key enzyme for inflammatory cytokine-induced angiogenesis. FASEB J. 2004; 18(2):300-10.
Kwok W E, Lo K K, Seo G S, Totterman S M S. A Double-turn Dual Phased Array Coil for MR Microscopic Imaging at 1.5 Tesla. In: Proceedings of ISMRM 6th Scientific Meeting and Exhibition, Sydney, Australia, 1998, p2027.
Lamson, D. W., & Brignall, M. S. (1999). Antioxidants in cancer therapy: Their actions and interactions with oncologic therapies. Alternative Medicine Review, 4, 304-329.
Lamson, D. W., & Brignall, M. S. (2000). Antioxidant and cancer therapy II: Quick reference guide. Alternative Medicine Review, 5, 152-163.
Lefaix, J. L, Delanian, S, and Dubray, B. [Physiopathology of human superficial radiation-induced fibrosis], C R Seances Soc Biol Fil. 191: 777-95, 1997.
Lefaix, J. L, Verola, 0, Brocheriou, C, and Daburon, F. Early changes in irradiated skeletal muscle: a histo-enzymological and immunocytochemical study, Br J Radiol Suppl. 19: 109-13, 1986.
Li L, Steinauer K K, Dirks A J, Husbeck B, Gibbs I, Knox S J. Radiation-induced cyclooxygenase 2 up-regulation is dependent on redox status in prostate cancer cells. Radiat Res. 2003 December; 160(6):617-21
Li P, Allen H, Banerjee S, Seshadri T. Characterization of mice deficient in interleukin-1 beta converting enzyme. J Cell Biochem. 1997 January; 64(1):27-32
Liang L, Hu D, Liu W, Williams J P, Okunieff P, Ding I.: Celecoxib reduces skin damage after radiation: selective reduction of chemokine and receptor mRNA expression in irradiated skin but not in irradiated mammary tumor. Am J Clin Oncol. 2003; 26(4):S114-21.
Liao C H, Sang S, Liang Y C, Ho C T, Lin J K. Suppression of inducible nitric oxide synthase and cyclooxygenase-2 in downregulating nuclear factor-kappa B pathway by Garcinol. Mol. Carcinog. 2004 Aug. 24
M. J. Trotter, D. J. Chaplin, and P. L. Olive, Use of a carbocyanine dye as a marker of functional vasculature in murine tumours, British Journal of Cancer 1989; 59:706-709.
MacKay, R. I, Niemierko, A, Goitein, M, and Hendry, J. H. Potential clinical impact of normal-tissue intrinsic radiosensitivity testing, Radiother Oncol. 46: 215-6, 1998.
Maier C M, Hsieh L, Yu F, Bracci P, Chan P H. Matrix metalloproteinase-9 and myeloperoxidase expression: quantitative analysis by antigen immunohistochemistry in a model of transient focal cerebral ischemia. Stroke. 2004; 35(5):1169-74.
Maisin J R.: Bacq and Alexander Award lecture—chemical radioprotection: past, present, and future prospects. Int J Radiat Biol. 1998 April; 73(4):443-50
Manetti F, Maccari L, Corelli F, Botta M: 3D QSAR models of interactions between beta-tubulin and microtubule stabilizing antimitotic agents (MSAA): a survey on taxanes and epothilones. Curr Top Med. Chem. 2004; 4(2):203-17.
Marini P, Belka C. Death receptor ligands: new strategies for combined treatment with ionizing radiation. Curr Med Chem Anti-Canc Agents. 2003; 3(5):334-42
Marks, L. B, Fan, M, Clough, R, Munley, M, Bentel, G, Coleman, R. E, Jaszczak, R, Hollis, D, and Anscher, M. Radiation-induced pulmonary injury: symptomatic versus subclinical endpoints, Int J Radiat Biol. 76: 469-75, 2000.
Marrogi A, Pass H I, Khan M, Metheny-Barlow L J, Harris C C, Gerwin B I. Human mesothelioma samples overexpress both cyclooxygenase-2 (COX-2) and inducible nitric oxide synthase (NOS2): in vitro antiproliferative effects of a COX-2 inhibitor. Cancer Res. 2000 Jul. 15; 60(14):3696-700
Marrogi A J, Travis W D, Welsh J A, Khan M A, Rahim H, Tazelaar H, Pairolero P, Trastek V, Jett J, Caporaso N E, Liotta L A, Harris C C. Nitric oxide synthase, cyclooxygenase 2, and vascular endothelial growth factor in the angiogenesis of non-small cell lung carcinoma. Clin Cancer Res. 2000 December; 6(12):4739-44.
McBride W H, Chiang C S, Olson J L, Wang C C, Hong J H, Pajonk F, Dougherty G J, Iwamoto K S, Pervan M, Liao Y P. A sense of danger from radiation. Radiat Res. 2004 July; 162(1):1-19.
Milas L. Cyclooxygenase-2 (COX-2) enzyme inhibitors as potential enhancers of tumor radioresponse. Semin Radiat Oncol. 2001 October; 11(4):290-9
Moore A H, Olschowka J A, Williams J P, Paige S L, O'Banion M K. Radiation-induced edema is dependent on cyclooxygenase 2 activity in mouse brain. Radiat Res. 2004; 161(2):153-60.
Murata, H., Kawano, S., Tsuji, S., Tsuji, M., Sawaoka, H., Kimura, Y., Shiozaki, H., Hori, M. (1999) Cyclooxygenase-2 overexpression enhances lymphatic invasion and metastasis in human gastric carcinoma. Am. J. Gastroenterol. 94, 451-455
Neta R, Keller J R, Ali N, Blanchette F, Dubois C M. Contrasting mechanisms of the myeloprotective effects of interleukin-1 against ionizing radiation and cytotoxic 5-fluorouracil. Radiat Res. 1996 May; 145(5):624-31.
Niemierko, A. and Goitein, M. Modeling of normal tissue response to radiation: the critical volume model, Int J Radiat Oncol Biol Phys. 25: 135-45, 1993.
Okunieff, P, Rubin, P, Williams, J, Finkelstein, J, and Ding, I. TGF-b1 is an important contributing factor in the development of radiation induced fibrosis, Int. J. Radiat. Oncol. Biol. Phys. 46:, 2000.
Olson J J, Beck D W, Warner D S, Coester H.: The role of new vessels and macrophages in the development and resolution of edema following a cortical freeze lesion in the mouse. J Neuropathol Exp Neurol. 1987; 46 (6):682-94
Orr G A, Verdier-Pinard P, McDaid H, Horwitz S B Mechanisms of Taxol resistance related to microtubules. Oncogene. 2003 Oct. 20; 22(47):7280-95
Papahadjopoulos, D., Allen, T. M., Gabizon, A., Mayhew, E., Matthay, K., Huang, S. K., Lee, K. D., Woodle, M. C., Lasic, D. D., Redeman, C., and Martin, F. J. Sterically stabilized liposomes: Improvements in pharmacokinetics and tumor therapeutic efficacy. Proc. Natl. Acad. Sci. U.S.A. 1991; 88, 11460-11464.
Petersen C, Baumann M, Petersen S, New targets for the modulation of radiation response—selective inhibition of the enzyme cyclooxygenase 2. Curr Med Chem Anti-Canc Agents. 2003 September; 3(5):354-9.
Potten C S, Booth D, Haley J D. Pretreatment with transforming growth factor beta-3 protects small intestinal stem cells against radiation damage in vivo. Br J. Cancer. 1997; 75(10):1454-9.
Pradilla G, Wang P P, Legnani F G, Ogata L, Dietsch G N, Tamargo R J. Prevention of vasospasm by anti-CD11/CD18 monoclonal antibody therapy following subarachnoid hemorrhage in rabbits. J. Neurosurg. 2004 July; 101(1):88-92.
Richter K K, Fagerhol M K, Carr J C, Winkler J M, Sung C C, Hauer-Jensen M. Association of granulocyte transmigration with structural and cellular parameters of injury in experimental radiation enteropathy. Radiat Oncol Investig. 1997; 5(6):275-82.
Richter W, Richter M, Warren W S, Merkle H, Andersen P, Adriany G, Ugurbil K. Functional magnetic resonance imaging with intermolecular multiple-quantum coherences. Magn Reson Imaging. 2000 June; 18(5):489-94.
Roh J L, Sung M W, Park S W, Heo D S, Lee D W, Kim K H. Celecoxib can prevent tumor growth and distant metastasis in postoperative setting. Cancer Res. 2004 May 1; 64(9):3230-5.
Rothwell, R. I., Kelly, S. A. and Joslin, C. A. Radiation pneumonitis in patients treated for breast cancer. Radiother. Oncol., 1985; 4, 9-14
Rubin, P, Constine, L. S, 3rd, Fajardo, L. F, Phillips, T. L, and Wasserman, T. H. EORTC Late Effects Working Group. Overview of late effects normal tissues (LENT) scoring system, Radiother Oncol. 35: 9-10, 199
Sandler A B, Dubinett S M. COX-2 inhibition and lung cancer. Semin Oncol. 2004 April; 31(2 Suppl 7):45-52
Sano, H., Kawahito, Y., Wilder, R. L., Hashiramoto, A., Mukai, S., Asai, K., Kimura, S., Kato, H., Kondo, M., Hla, T. (1995) Expression of cyclooxygenase-1 and -2 in human colorectal cancer. Cancer Res. 55, 3785-3789
Schumacher J, Binkowski K, Dendorfer A, Klotz K F. Organ-specific extravasation of albumin-bound Evans blue during nonresuscitated hemorrhagic shock in rats. Shock. 2003; 20(6):565-8.
Segal S S, Brett S E, Sessa W C Codistribution of NOS and caveolin throughout peripheral vasculature and skeletal muscle of hamsters. Am J. Physiol. 1999 September; 277(3 Pt 2):H1167-77.
Sheng, H., Shao, J., Morrow, J. D., Beauchamp, R. D., DuBois, R. N.: Modulation of apoptosis and Bcl-2 expression by prostaglandin E2 in human colon cancer cells. Cancer Res. 58, 362-366, 1998
Sheng, H., Shao, J., Washington, M. K., DuBois, R. N.: Prostaglandin E2 increases growth and motility of colorectal carcinoma cells. J. Biol. Chem. 276, 18075-18081, 2001
Stefan Danner and Joel G. Belasco: T7 phage display: A novel genetic selection system for cloning RNA-binding proteins from cDNA libraries. Proc Natl Acad Sci USA. 2001; 98 (23): 12954-12959
Sunada Y, Ohi H, Hase A, Ohi H, Hosono T, Arata S, Higuchi S, Matsumura K, Shimizu T. Transgenic mice expressing mutant caveolin-3 show severe myopathy associated with increased nNOS activity. Hum Mol. Genet. 2001; 10(3):173-8.
Taghian A G, Assaad S I, Niemierko A, Kuter I, Younger J, Schoenthaler R, et al. Risk of pneumonitis in breast cancer patients treated with radiation therapy and combination chemotherapy with paclitaxel. J Natl Cancer Inst 2001; 93:1806-11
Totterman S M, Herberger R, Miller R, Rubens D J, Blebea J S. Two-piece wrist surface coil. AJR 1991; 156:343-344.
Tsuji, S., Kawano, S., Tsujii, M., Michida, T., Masuda, E., Gunawan, E. S., Hori, M.: Mucosal microcirculation and angiogenesis in gastrointestinal tract. Nippon Rinsho. 56, 2247-2252 1998
Tsuji, S., Tsujii, M., Kawano, S., Hori, M. (2001) Cyclooxygenase-2 upregulation as a perigenetic change in carcinogenesis. J. Exp. Clin. Cancer Res. 20, 117-129
Tucker, S. L., Geara, F. B., Peters, L. J. and Brock, W. A. How much could the radiotherapy dose be altered for individual patients based on a predictive assay of normal-tissue radiosensitivity? Radiother. Oncol., 1996; 38, 103-113.
Verola, O, Lefaix, J. L, Daburon, F, and Brocheriou, C. Vascular damage after acute local irradiation: a light and electron microscope study, Br J Radiol Suppl. 19: 104-8, 1986.
Vijayalaxmi, Reiter R J, Tan D X, Herman T S, Thomas C R Jr. Melatonin as a radioprotective agent: a review. Int J Radiat Oncol Biol Phys. 2004; 59(3):639-53
Volonte D, Peoples A J, Galbiati F. Modulation of myoblast fusion by caveolin-3 in dystrophic skeletal muscle cells: implications for Duchenne muscular dystrophy and limb-girdle muscular dystrophy-1C. Mol Biol Cell. 2003 October; 14(10):4075-88. Epub 2003 Aug. 7
Vozenin-Brotons, M. C, Gault, N, Sivan, V, Tricaud, Y, Dubray, B, Clough, K, Cosset, J. M, Lefaix, J. L, and Martin, M. Histopathological and cellular studies of a case of cutaneous radiation syndrome after accidental chronic exposure to a cesium source, Radiat Res. 152: 332-7, 1999.
Vujaskovic Z, Feng Q F, Rabbani Z N, Samulski T V, Anscher M S, Brizel D M. Assessment of the protective effect of amifostine on radiation-induced pulmonary toxicity. Exp Lung Res. 2002 October-November; 28(7):577-90.
Wadleigh, D. J., Herschman, H. R. (1999) Transcriptional regulation of the cyclooxygenase-2 gene by diverse ligands in murine osteoblasts. Biochem. Biophys. Res. Commun. 264, 865-870
Wang H-B.; Fang J.; Zheng Q-Y. Inhibitory effect of esculentoside A on prostaglandin E2 production from murine peritoneal macrophages and rabbit synovial cells in vitro Mediators of Inflammation, 1 Feb. 1997, vol. 6, no. 1, pp. 22-24(3)
Wang J, Albertson C M, Zheng H, Fink L M, Herbert J M, Hauer-Jensen M. Short-term inhibition of ADP-induced platelet aggregation by clopidogrel ameliorates radiation-induced toxicity in rat small intestine. Thromb Haemost. 2002 January; 87(1):122-8.
Wang J, Zheng H, Hauer-Jensen M: Influence of Short-Term Octreotide Administration on Chronic Tissue Injury, Transforming Growth Factor beta (TGF-beta) Overexpression, and Collagen Accumulation in Irradiated Rat Intestine. J Pharmacol Exp Ther. 2001 April; 297(1):35-42
Weiss J F, Landauer M R Protection against ionizing radiation by antioxidant nutrients and phytochemicals. Toxicology. 2003 Jul. 15; 189(1-2):1-20
Weiss J F. Pharmacologic approaches to protection against radiation-induced lethality and other damage. Environ Health Perspect. 1997 December; 105 Suppl 6:1473-8.
Williams, C. S., DuBois, R. N. (1996) Prostaglandin endoperoxide synthase: why two isoforms?. Am. J. Physiol. 270,G393-G40
Witton C J, Hawe S J, Cooke T G, Bartlett J M Cyclooxygenase 2 (COX2) expression is associated with poor outcome in ER-negative, but not ER-positive, breast cancer. Histopathology. 2004 July; 45(1):47-54
Xiao Z Y, Zhang J P, Lu F, Zhang D Z, Zheng Q Y. Effect of esculentoside A on cellular adhesion. Yao Xue Xue Bao. 2003 October; 38(10):728-30.
Xiao Z Y, Zheng Q Y, Jiang Y Y, Zhou B, Yin M, Wang H B, Zhang J P. Effects of esculentoside A on production of interleukin-1, 2, and prostaglandin E2. Acta Pharmacol Sin. 2004 June; 25(6):817-21.
Xiao Z Y, Zheng Q Y, Zhang J P, Jiang Y Y, Yi Y H: Effect of esculentoside A on autoimmunity in mice and its possible mechanisms. Acta Pharmacol Sin. 2002 July; 23(7):638-44.
Xu L, Frederik P, Pirollo K F, Tang W H, Rait A, Xiang L M, Huang W, Cruz I, Yin Y, Chang E H. Self-assembly of a virus-mimicking nanostructure system for efficient tumor-targeted gene delivery. Hum Gene Ther. 2002 Feb. 10; 13(3):469-81
Xu L, Huang C C, Huang W, Tang W H, Rait A, Yin Y Z, Cruz I, Xiang L M, Pirollo K F, Chang E H. Systemic tumor-targeted gene delivery by anti-transferrin receptor scFv-immunoliposomes. Mol Cancer Ther. 2002 March; 1(5):337-46.
Xu L, Pirollo K F, Chang E H. Transferrin-liposome-mediated p53 sensitization of squamous cell carcinoma of the head and neck to radiation in vitro. Hum Gene Ther. 1997 Mar. 1; 8(4):467-75
Xue, Y. W., Zhang, Q. F., Zhu, Z. B., Wang, Q., Fu, S. B. (2003) Expression of cyclooxygenase-2 and clinicopathologic features in human gastric adenocarcinoma. World J. Gastroenterol. 9, 250-253
Yi Y, Wang Z: Study of effective components of Phytolacca esculent. Chinese Herb Medicine 1984, 15 (2): 7-11
Yixin Chen, XueMing Xu, Shuigen Hong, Jinguo Chen, Ningfei Liu, Charles B. Underhill, Karen Creswell and Lurong Zhang: RGD-Tachyplesin inhibits tumor growth. Cancer Res. 2001; 61: 2434-2438
Zaghloul M S, Dorie M J, Kallman R F. Interleukin-1 modulatory effect on the action of chemotherapeutic drugs and localized irradiation of the lip, duodenum, and tumor. Int J Radiat Oncol Biol Phys. 1993 Jun. 15; 26(3):417-25.
Zalipsky, S. Chemistry of polyethylene glycol conjugates with biologically active molecules. Adv. Drug Delivery Rev. 1995; 16, 157-182.
Zawadzka M, Kaminska B. A novel mechanism of FK506-mediated neuroprotection: Downregulation of cytokine expression in glial cells. Glia. 2004 July 27
Zhang, S.; Liang, X.; Danielsen, M. Role of the c terminus of the glucocorticoid receptor in hormone binding and agonist/antagonist discrimination. Mol Endocrinol 1996, 10(1), 24-34.
Zhang, S.; Lu, J.; Iyama, K.; Lo, S. C.; Danielsen, M. A simplified method for large scale quantification of transcriptional activity and its use in studies of steroids and steroid receptors. J Recept Signal Transduct Res 2001, 21(1), 71-84.
Zhong J, Chen Z, Kwok E In vivo intermolecular double-quantum imaging on a clinical 1.5 T MR scanner. Magn Reson Med. 2000 March; 43(3):335-41
Zimmermann J S, Kimmig B. Pharmacological management of acute radiation morbidity. Strahlenther Onkol. 1998 November; 174 Suppl 3:62-5
Zubova, S. G, Crechet, F, Gault, N, Danilov, A. O, Vozenin, M. K, Lefaix, J. L, Daburon, F, Martin, M, and Okulov, V. B. Effect of ionizing radiation on transforming growth factor-beta expression, Biull Eksp Biol Med. 126: 529-33, 1998.

1. A method of reducing radiation damage in a subject comprising administering to the subject an effective amount of a water soluble COX-2 inhibitor.

2. The method of claim 1, wherein the radiation damage is caused by radiation therapy.

3. The method of claim 2, wherein the radiation therapy is used to treat cancer.

4. The method of claim 1, wherein the radiation damage is caused by nuclear radiation.

5. The method of claim 1, wherein the radiation is caused by a weapon.

6. The method of claim 1, wherein the water soluble COX-2 inhibitor is a saponin.

7. The method of claim 6, wherein the saponin is esculentoside A (EsA) or a COX-2 inhibiting derivative thereof.

8. A method of inhibiting COX-2 in a subject comprising administering to the subject intraarticularly, intravenously, or transdermally a water soluble COX-2 inhibitor.

9-10. (canceled)

11. The method of claim 8, wherein the COX-2 inhibitor is a saponin.

12. The method of claim 11, wherein the saponin is EsA or a COX-2 inhibiting derivative thereof.

13. A method of inhibiting a cytokine in a subject comprising administering to the subject intraarticularly, intravenously, or transdermally a water soluble COX-2 inhibitor.

14. The method of claim 13, wherein the cytokine is selected from the group consisting of IL1, IL6, TNFα, TGFβ, VEGF, and MCP1 or any combination thereof.

15. The method of claim 13, wherein the COX-2 inhibitor is a saponin.

16. The method of claim 15, wherein the saponin is EsA or a cytokine inhibiting derivative thereof.

17-24. (canceled)

25. A method of inhibiting PGE2 in a subject comprising administering to the subject intrarticularly, transdermally, or intravenously a water soluble COX-2 inhibitor.

26. A method of inhibiting nitric oxide (NO) in a subject comprising administering to the subject intraarticularly, transdermally, or intravenously a water soluble COX-2 inhibitor.

27. A method of inhibiting angiogenesis in a subject comprising administering to the subject a water soluble COX-2 inhibitor.

28. A method of inhibiting brain edema in a subject comprising administering to the subject an effective amount of a water soluble COX-2 inhibitor.

29. The method of claim 28, wherein the COX-2 inhibitor is a saponin.

30. The method of claim 29, wherein the saponin is EsA or an edema inhibiting derivative thereof.

31. The method of claim 28, wherein the brain edema is radiation induced.

32. A composition comprising a COX-2 inhibiting derivative of EsA.
